(19) United States
(12) Patent Application Publication
(10) Pub. No.: US 2024/0240237 A1
(43) Pub. Date: Jul. 18, 2024

(54) LINKED AMPLIFICATION TETHERED WITH EXPONENTIAL RADIANCE

(71) Applicant: CALIFORNIA INSTITUTE OF TECHNOLOGY, Pasadena, CA (US)

(72) Inventors: Long CAI, Pasadena, CA (US); Chee Huat ENG, Pasadena, CA (US); Lincoln OMBELETS, Pasadena, CA (US)

(21) Appl. No.: 18/563,676

(22) PCT Filed: Mar. 24, 2022
(86) PCT No.: PCT/US2022/021826

§ 371 Date: Nov. 22, 2023

Related U.S. Application Data
(60) Provisional application No. 63/192,554, filed on May 24, 2021.

(51) Int. Cl. C12Q 1/682; C12Q 1/6841

(52) U.S. Cl. CPC C12Q 1/682; C12Q 1/6841

(57) ABSTRACT

Disclosed herein is a composition for linked amplification tethered with exponential radiance for signal amplification. Also disclosed herein, is a kit for linked amplification tethered with exponential radiance for signal amplification. Also disclosed herein, is a method linked amplification tethered with exponential radiance for signal amplification.

### CROSS-REFERENCE TO RELATED APPLICATIONS

This application claims the benefit of U.S. Provisional Patent Application No. 63/192,554, filed May 24, 2021. The contents of the above-referenced application are hereby incorporated by reference in their entirety.

### STATEMENT REGARDING FEDERALLY-SPONSORED RESEARCH

This invention was made with government support under Grant No. HD075605 awarded by the National Institutes of Health. The government has certain rights in the invention.

### FIELD OF INVENTION

The present disclosure provides methods, compositions, kits for scalable signal amplification of amplicons that can be applied to multiplexed imaging to profile biological samples.

### BACKGROUND

Transcription profiling of cells is valuable for many purposes. Microscopic imaging resolving multiple mRNAs in single cells can provide information regarding transcript abundance and localization, which are important for understanding the molecular basis of cell identify and developing treatment for diseases. Molecular profiling such as transcriptomic profiling of biological samples is valuable for various purposes. For example, it could allow one to assess gene expression levels to detect and identify abnormal growth states such as cancers.

Techniques such as qPCR and microarrays have been useful, but they do not reach single molecule sensitivity. Next generation sequencing, on the other hand, involves amplification of a sample and reverse transcription of mRNA that can introduce biases and inaccuracies in quantification. Moreover, sample preparation and sequencing can be time-consuming and costly. Despite the fact that imaging has been used for mRNA transcripts quantification, it is limited to a few hundred genes. Many scientific questions become accessible if thousands of genes or even the whole transcriptome can be quantified.

What is needed are better methods and systems for carrying out imaging based transcriptomic profiling at single molecule sensitivity with high accuracy in a time efficient manner.

### SUMMARY

The present disclosure provides methods, compositions, kits, and for linked amplification tethered with exponential radiance (LANTERN) precise and deterministic signal amplification. LANTERN allows a scalable signal amplification of amplicons that can be applied to multiplexed imaging to profile biological samples. This disclosure sets forth compositions and kits, in addition to making and using the same, and other solutions to problems in the relevant field.

In some embodiments, there is provided a composition for amplification tethered with exponential radiance, comprising a plurality of probes, wherein the composition comprises: one or more primary probes capable of binding one or more targets, wherein each primary probe comprises one or more secondary probe binding sites and optionally one or more readout probe binding sites. In certain embodiments, the composition comprises one or more secondary probes, each capable of binding the primary probe, wherein each secondary probe comprises one or more tertiary probe binding sites or one or more readout probe binding sites. In certain embodiments, the composition optionally comprises one or more tertiary probes, each capable of binding to the secondary probe, wherein each tertiary probe comprises one or more quaternary probe binding sites or one or more readout probe binding sites. In certain embodiments, the composition optionally comprises one or more quaternary probes, each capable of binding to the tertiary probe, wherein each quaternary probe comprises one or more readout probe binding sites. In certain embodiments, the composition comprises one or more readout probes capable of binding to a binding site on the one or more primary, secondary, tertiary, or quaternary probes and capable of being detected. In certain embodiments, the composition comprises one or more molecules capable of stabilizing one or more primary, secondary, tertiary, or quaternary probes when or after the probe is hybridized or after the probe is hybridized.

In some embodiments, there is provided a kit for linked amplification tethered with exponential radiance, comprising a plurality of probes, wherein the composition comprises: wherein each primary probe comprises one or more secondary probe binding sites and optionally one or more readout probe binding sites. In certain embodiments, the kit comprises one or more secondary probes, each capable of binding the primary probe, wherein each secondary probe comprises one or more tertiary probe binding sites or one or more readout probe binding sites. In certain embodiments, the kit optionally comprises one or more tertiary probes, each capable of binding to the secondary probe, wherein each tertiary probe comprises one or more quaternary probe binding sites or one or more readout probe binding sites. In certain embodiments, the kit optionally comprises one or more quaternary probes, each capable of binding to the tertiary probe, wherein each quaternary probe comprises one or more readout probe binding sites. In certain embodiments, the kit comprises one or more readout probes capable of binding to a binding site on the one or more primary, secondary, tertiary, quaternary probes and capable of being detected. In certain embodiments, the kit comprises one or more molecules capable of stabilizing one or more primary, secondary, tertiary, or quaternary probes when or after the probe is hybridized or after the probe is hybridized.

In some embodiments, there is provided a method for linked amplification tethered with exponential radiance, comprising a method for ligation-amplifying fluorescence in situ hybridization, comprising steps of: contacting a sample with one or more primary probes that bind one or more targets, wherein each primary probe hybridizes to the target. In certain embodiments, the method comprises hybridizing one or more secondary probes to the primary probes; wherein each secondary probe comprises one or more tertiary probe binding sites or one or more readout probe binding sites. In certain embodiments, the method optionally comprises hybridizing one or more tertiary probes to at least one secondary probe; and wherein each tertiary probe comprises one or more quaternary probes or one or more readout probe binding sites. In some embodiments, the method comprises optionally, hybridizing one or more quaternary probe to at least one tertiary probe, wherein each quaternary probe comprises one or more readout probe binding sites. In some embodiments, the method comprises stabilizing one or more primary, secondary, tertiary, or quaternary probes during or after steps (i)-(iv). In certain embodiments, the method comprises hybridizing readout probes capable of detection to the one or more readout probe binding sites. In certain embodiments, the method comprises imaging the cell after step (vi) so that the interaction of the primary probe to the nucleic acids is detected. In certain embodiments, the method comprises optionally repeating the contacting and imaging steps, each time with a new plurality of detectably labeled readout probes, wherein at least one readout probe for one target differs from at least one other readout probes for the same target in their detectable moieties, so that a target in the sample is described by a barcode, and can be differentiated from another target in the sample by a difference in their barcodes. In some embodiments, any of the preceeding embodiments are repeated either individually or in any combination thereof.

In some embodiments, the methods are used to generate probes for use in an efficient and scalable signal amplification method that can be applied to multiplexed imaging. In certain embodiments, the methods are used to generate probes for use in RNA and DNA sequential Fluorescence In Situ Hybridization (seqFISH). In certain embodiments, the methods are used to generate probes for use in immunofluorescence studies.

In contrast to other methods such as hybridization chain reaction (HCR), which only allows amplification of several amplicons at once, which is extremely time consuming when imaging tens or hundreds of species from one sample, the methods disclosed herein regarding the primary, secondary, tertiary, quaternary, and readout probes are significantly simpler and less expensive to synthesize than other chemical modifications and are easily compatible with existing enzymatic probe synthesis protocols.

### BRIEF DESCRIPTIONS OF THE DRAWINGS

FIG. 1. Left: Overview of LANTERN scheme, showing two different target sequences as an example. Different colors represent the orthogonal sequences used to amplify each distinct target (shown as ‘A’ and ‘B’), and the orthogonal readout probe binding sites (shown as ‘C’). One round of amplification is shown, corresponding to 2*2*4=16-fold amplification compared to directly reading out the primary probe. Right: Top: Widefield RNA FISH against Eef2 with two probe sets in different colors, shown as magenta and cyan in NIH3T3 cells. (A) conventional smFISH, exposure time 100 ms. (B) 6 rounds of LANTERN, exposure time 1 ms. Bottom: Confocal RNA LANTERN (6 rounds) against Eef2 in NIH3T3 cells. (C) First hybridization (D) Rehybridization #12, corresponding to ˜10 hours real time, showing stability of amplified signal over repeated formamide stripping and rehybridization. All images taken with 63× oil objective lens unless otherwise noted.

FIG. 2. Left: Pairwise colocalization heatmap for a pooled test of 60 LANTERN amplifiers. 2D colocalization was estimated by searching maximum-projected images for local maxima in one image in a 3-pixel box around local maxima in a second image. Off-diagonal squares corresponding to adjacent amplifiers (for example: 1 and 2, 3 and 4) have high colocalization because pairs of amplifiers were targeted to the same gene for this experiment. Center: Scatterplot showing correlation between number of dots detected by smFISH (horizontal axis) and LANTERN (vertical axis) on the same genes. There were two amplifiers assigned to each gene. Right: Scatterplot showing correlation between number of dots detected by the first amplifier (horizontal axis) with the number detected by the second amplifier (vertical axis) for the same gene.

FIG. 3. Widefield images of RNA FISH for Eif4g1 in NIH3T3 cells showing colocalization among two orthogonal amplified sequences (A and B) and conventional smFISH (C) for the same gene. Exposure times 40 ms (A and B) and 400 ms (C). (D) shows all three channels at once. DAPI nuclear stain shown in gray.

FIG. 4. Confocal images of telomere DNA FISH in NIH3T3 cells (cyan). Left: unamplified telomere DNA FISH, 500 ms exposure time. Right: LANTERN-amplified telomere DNA FISH, 500 ms exposure time. Images are shown with identical contrast. DAPI nuclear stain is shown in gray.

FIG. 5. Widefield images of NIH3T3 cells stained with DNA-conjugated antibodies before (A and C) and after (B and D) LANTERN amplification. A and B stained with anti-Lamin B and DNA-conjugated secondary antibody showing nuclear lamina staining. Exposure time 200 ms (A) and 10 ms (B). C and D stained with anti-TIMM44 and DNA-conjugated secondary antibody showing mitochondrial staining. Exposure time 200 ms (C) and 20 ms (D).

FIG. 6. A-C: Confocal images of RNA FISH for Eef2 in human breast cancer biopsy sample. (A) Before clearing, showing high background, exposure 1s. (B) After clearing, showing reduced background, exposure 1s. (C) After clearing and LANTERN amplification, showing brighter and clearer dots. Exposure 100 ms. (D-E) Confocal images of RNA FISH for Eef2 in adult mouse brain section. D: smFISH signal (cyan), exposure 4s. (E) Different brain sample after clearing and LANTERN amplification (cyan), exposure 50 ms. (F-G) Confocal images of RNA FISH for Notch1 (cyan and magenta) and Ling (green) introns in whole-mount chicken embryo. (F) unamplified signal, hardly visible at this threshold, exposure 1s. (G) signal after LANTERN, exposure 1s. All samples of the same type are displayed with identical contrast levels, and DAPI staining of nuclei is gray in lower panels.

FIG. 7: Representative amplifiers which amplify the fluorescent signals and highly colocalize.

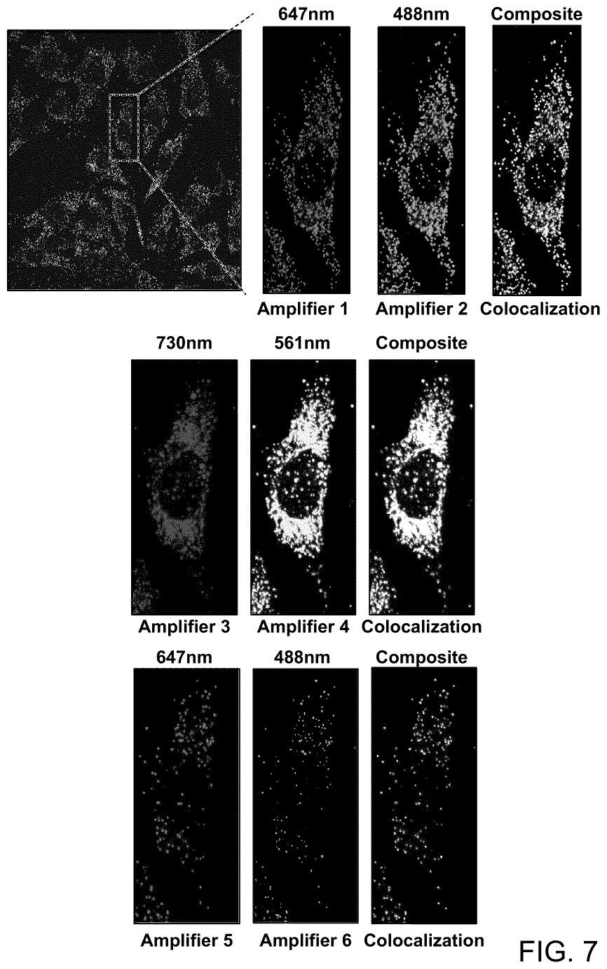

FIG. 8: LANTERN is highly specific. Most amplified signals come from correctly bound padlock probes. Images are displayed in the same contrast to demonstrate the higher intensity fluorescent spots from LANTERN in comparison to non-amplified fluorescent spots.

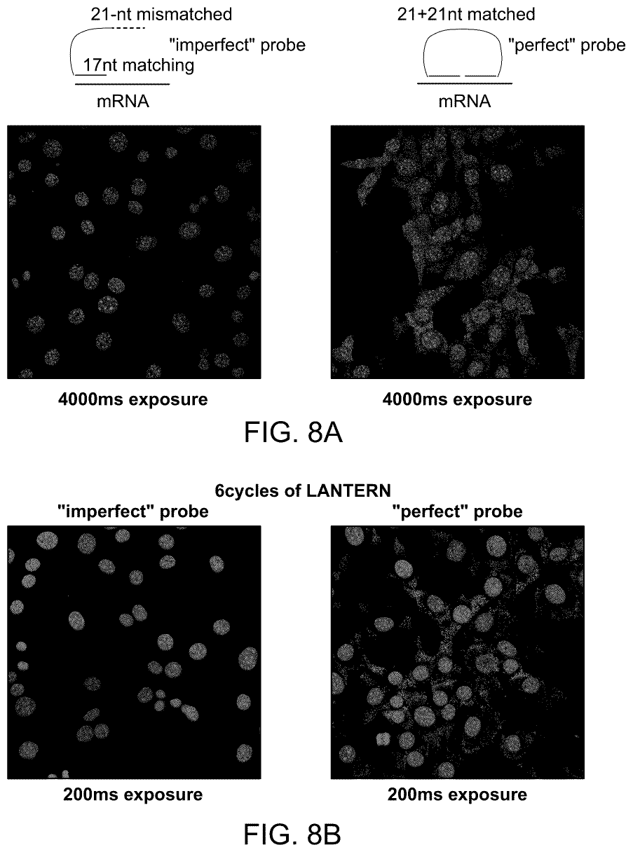

FIG. 9: LANTERN overcomes the autofluorescence and lipofuscin in Alzheimer's disease human brain section. (A) LANTERN amplified fluorescent spots for gene Eef2 are retained after thresholding away the lipofuscin intensity counts, indicating amplified fluorescent spots are much brighter. (B) In comparison to non-amplified, single molecule FISH fluorescent spots, thresholding removes the transcripts spots while lipofuscin still strongly remains. Images are displayed in the same contrast to demonstrate the brighter signals after LANTERN amplification.

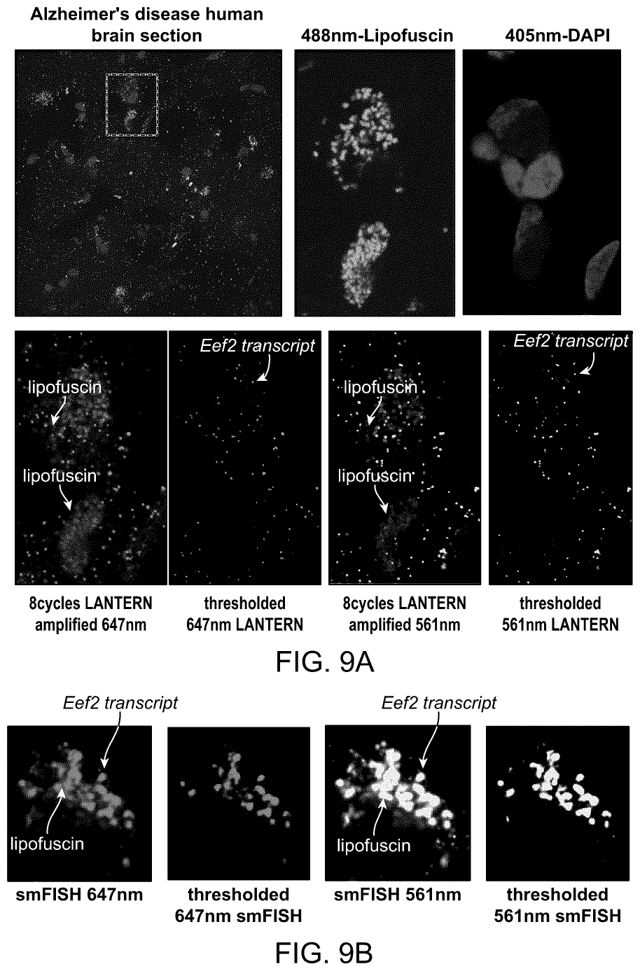

FIG. 10: LANTERN amplifies fluorescent signals in perfused, overnight paraformaldehyde fixed mouse brain tissue section. Top panel's contrast is 10× lower than the middle panel which has the same contrast as the bottom panel. This shows that fluorescent signals in the same single cells are amplified after 6 cycles of LANTERN.

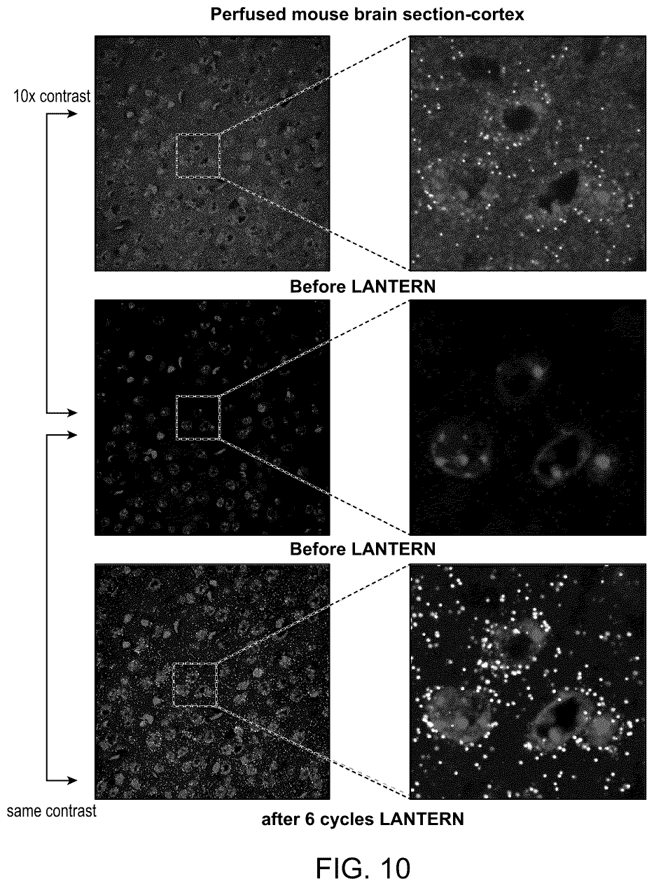

FIG. 11: Quantitative assessment of LANTERN in highly multiplexed seqFISH+ experiment. (A). Top panel: example of one of the ‘pseudocolors’ hybridization in seqFISH+ experiment before LANTERN with exposure time of 5s in all fluorescent channels. Bottom panel: LANTERN amplified images with 200 ms exposure in 647 nm channel, 300 ms exposure in 561 nm channel, and 400 ms exposure in 488 nm channel. Images in the same fluorescent channel are displayed with the same contrast. (B) LANTERN amplification folds in each fluorescent channel. 647 nm, 561 nm and 488 nm fluorescent channels showed an amplification fold of 76.03, 59.49, and 59.82 respectively after 10 cycles of LANTERN, relative to smFISH. (C) Comparison of seqFISH+ experiment profiling 3,000 genes with LANTERN to bulk RNA-seq measurement. 45 pairs of unique LANTERN amplifiers are used in this experiment. The result shows a good Pearson's correlation coefficient of 0.73 to RNA-seq measurement.

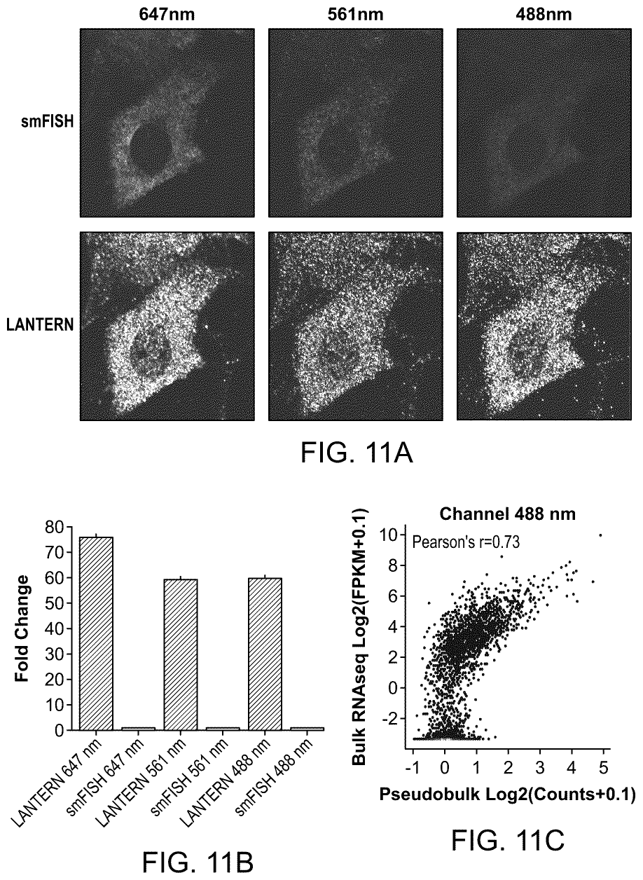

FIG. 12: Alternative schemes of LANTERN. (A) A pair of split amplifiers could be used to hybridize to the padlock primary probes in this case or an inverted padlock primary probes, in other cases. Then an enzymatic ligase is used to ligate the split amplifiers, incorrect bound split amplifiers will not be ligated and will be washed off with the high concentration formamide wash. Next, a split tertiary amplifier pair is hybridized to the ligated secondary amplifiers, followed by enzymatic ligation and harsh formamide wash. This cycle is repeated by iterating split amplifier hybridization, enzymatic ligation, and formamide harsh wash until a desirable amplification fold is achieved. Finally, the amplified scaffold can be detected with fluorophore conjugated readout oligonucleotides. (B) An optional design of split amplifiers which contains additional sequences which could be further ligate with a ligating enzyme through a splint sequence, further stabilizing the correctly bound amplifiers. (C) Amplified sample of Eef2 transcripts detected with split amplifiers in 647 nm fluorescent channel.

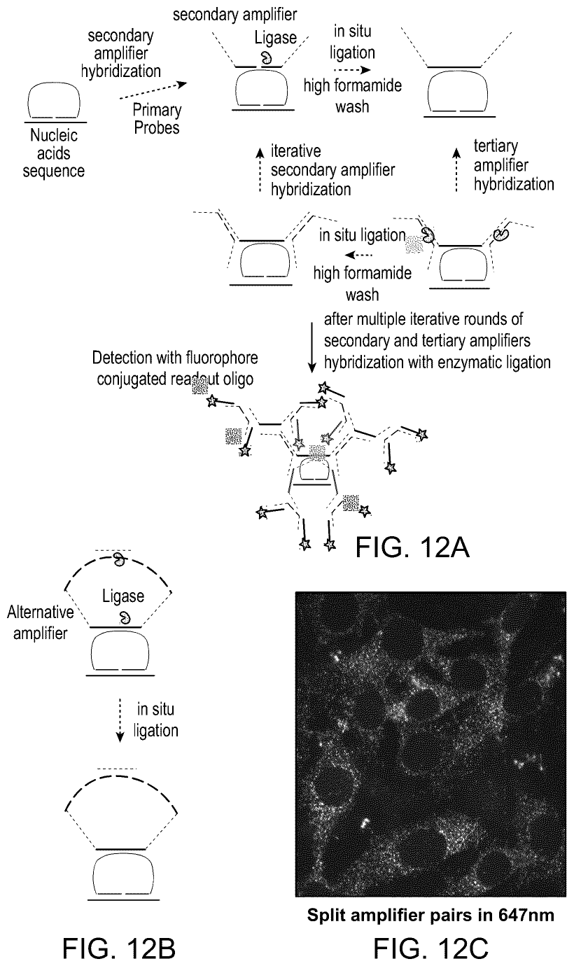

### DETAILED DESCRIPTION

The following description is presented to enable one of ordinary skill in the art to make and use the disclosed subject matter and to incorporate it in the context of applications. Various modifications, as well as a variety of uses in different applications, will be readily apparent to those skilled in the art, and the general principles defined herein may be applied to a wide range of embodiments. Thus, the present disclosure is not intended to be limited to the embodiments presented, but is to be accorded the widest scope consistent with the principles and novel features disclosed herein.

### Definitions

Unless otherwise noted, terms are to be understood according to conventional usage by those of ordinary skill in the relevant art.

As used herein, the terms “approximately” or “about” in reference to a number are generally taken to include numbers that fall within a range of 5%. 10%. 15%, or 20% in either direction (greater than or less than) of the number unless otherwise stated or otherwise evident from the context (except where such number would be less than 0% or exceed 100% of a possible value).

As used herein, the term “LANTERN” is an acronym that refers to linked amplification tethered with exponential radiance.

The term “oligonucleotide” refers to a polymer or oligomer of nucleotide monomers, containing any combination of nucleobases, modified nucleobases, sugars, modified sugars, phosphate bridges, or modified bridges. Oligonucleotides can be of various lengths. In particular embodiments, oligonucleotides can range from about 2 to about 1000 nucleotides in length. In various related embodiments, oligonucleotides, single-stranded, double-stranded, and triple-stranded, can range in length from about 4 to about 10 nucleotides, from about 10 to about 50 nucleotides, from about 20 to about 50 nucleotides, from about 15 to about 30 nucleotides, from about 20 to about 30 nucleotides in length. In some embodiments, the oligonucleotide is from about 9 to about 39 nucleotides in length. In some embodiments, the oligonucleotide is at least 4 nucleotides in length. In some embodiments, the oligonucleotide is at least 5 nucleotides in length. In some embodiments, the oligonucleotide is at least 6 nucleotides in length. In some embodiments, the oligonucleotide is at least 7 nucleotides in length. In some embodiments, the oligonucleotide is at least 8 nucleotides in length. In some embodiments, the oligonucleotide is at least 9 nucleotides in length. In some embodiments, the oligonucleotide is at least 10 nucleotides in length. In some embodiments, the oligonucleotide is at least 11 nucleotides in length. In some embodiments, the oligonucleotide is at least 12 nucleotides in length. In some embodiments, the oligonucleotide is at least 15 nucleotides in length. In some embodiments, the oligonucleotide is at least 20 nucleotides in length. In some embodiments, the oligonucleotide is at least 25 nucleotides in length. In some embodiments, the oligonucleotide is at least 30 nucleotides in length. In some embodiments, the oligonucleotide is a duplex of complementary strands of at least 18 nucleotides in length. In some embodiments, the oligonucleotide is a duplex of complementary strands of at least 21 nucleotides in length.

As used herein, the term “probe” or “probes” refers to any molecules, synthetic or naturally occurring, that can attach themselves directly or indirectly to a molecular target (e.g., an mRNA sample, DNA molecules, protein molecules, RNA and DNA isoform molecules, single nucleotide polymorphism molecules, and etc.). For example, a probe can include a nucleic acid molecule, an oligonucleotide, a protein (e.g., an antibody or an antigen binding sequence), or combinations thereof. For example, a protein probe may be connected with one or more nucleic acid molecules to for a probe that is a chimera. As disclosed herein, in some embodiments, a probe itself can produce a detectable signal. In some embodiments, a probe is connected, directly or indirectly via an intermediate molecule, with a signal moiety (e.g., a dye or fluorophore) that can produce a detectable signal.

As used herein, the term “binding sites” refer to a portion of a probe where other molecules may bind to the probe. In certain embodiments, the binding sites of a probe bind to another molecule through a non-covalent interaction.

As used herein, the term “sample” refers to a biological sample obtained or derived from a source of interest, as described herein. In some embodiments, a source of interest comprises an organism, such as an animal or human. In some embodiments, a biological sample comprises biological tissue or fluid. In some embodiments, a biological sample is or comprises bone marrow; blood; blood cells; ascites; tissue or fine needle biopsy samples; cell-containing body fluids; free floating nucleic acids; sputum; saliva; urine; cerebrospinal fluid, peritoneal fluid; pleural fluid; feces; lymph; gynecological fluids; skin swabs; vaginal swabs; oral swabs; nasal swabs; washings or lavages such as a ductal lavages or broncheoalveolar lavages; aspirates; scrapings; bone marrow specimens; tissue biopsy specimens; surgical specimens; feces, other body fluids, secretions, and/or excretions; and/or cells therefrom, etc. In some embodiments, a biological sample is or comprises cells obtained from an individual. In some embodiments, a sample is a “primary sample” obtained directly from a source of interest by any appropriate means. For example, in some embodiments, a primary biological sample is obtained by methods selected from the group consisting of biopsy (e.g., fine needle aspiration or tissue biopsy), surgery, collection of body fluid (e.g., blood, lymph, feces etc.), etc. In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example nucleic acids or proteins extracted from a sample or obtained by subjecting a primary sample to techniques such as amplification or reverse transcription of mRNA, isolation and/or purification of certain components, etc. In some embodiments, the term “sample” refers to a nucleic acid such as DNA, RNA, transcripts, or chromosomes. In some embodiments, the term “sample” refers to nucleic acid that has been extracted from the cell.

As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and/or chemical phenomena.

As disclosed herein, the term “label” generally refers to a molecule that can recognize and bind to specific target sites within a molecular target in a cell. For example, a label can comprise an oligonucleotide that can bind to a molecular target in a cell. The oligonucleotide can be linked to a moiety that has affinity for the molecular target. The oligonucleotide can be linked to a first moiety that is capable of covalently linking to the molecular target. In certain embodiments, the molecular target comprises a second moiety capable of forming the covalent linkage with the label. In particular embodiments, a label comprises a nucleic acid sequence that is capable of providing identification of the cell which comprises or comprised the molecular target. In certain embodiments, a plurality of cells is labelled, wherein each cell of the plurality has a unique label relative to the other labelled cells.

As disclosed herein, the term “barcode” generally refers to a nucleotide sequence of a label produced by methods described herein. The barcode sequence typically is of a sufficient length and uniqueness to identify a single cell that comprises a molecular target.

As disclosed herein, the term “linked” refers to a covalent bond or non-covalent interaction between two molecules. In particular, a type of non-covalent interaction is a hybridization.

As disclosed herein, the term “cis-ligated” generally refers to the ligation of an oligonucleotide 5′ to 3′ on the same oligonucleotide. In certain embodiments, “cis-ligated” refers to the ligation of the ends of an oligonucleotide together. In certain embodiments, “cis ligated” refers to the ligation of one end of the oligonucleotide to a nucleotide at any place before the other end of the oligonucleotide.

As disclosed herein, the term “trans-ligated” generally refers to the ligation of an oligonucleotide 5′ to 3′ to a different oligonucleotide. In certain embodiments, “trans-ligated” refers to the ligation of the end of one oligonucleotide together to the end of another nucelotide. In certain embodiments, “trans-ligated” refers to the ligation of one end of the oligonucleotide to a nucleotide at any nucleotide on a different oligonucleotide.

As disclosed herein, the term “splint probe” or “splint” or “splint sequence” refers to a probe that is complementary to another probe that hybridizes or binds to the complementary probe, the probes are not covalently linked to each other. In certain embodiments, the term “splint probe” uses the definition and techniques of Lohman et al. Efficient DNA ligation in DNA-RNA hybrid helices by Chlorella virus DNA ligase. Nucleic Acid Research, 2014, vol. 42 No. 3 1831-1844, incorporated by reference in its entirety.

### EMBODIMENTS

The disclosure herein sets forth embodiments for a composition for linked amplification tethered with exponential radiance, comprising a plurality of probes.

The disclosure herein sets forth embodiments for a kit for linked amplification tethered with exponential radiance, comprising a plurality of probes.

The disclosure herein sets forth embodiments for a method for linked amplification tethered with exponential radiance, comprising a plurality of probes.

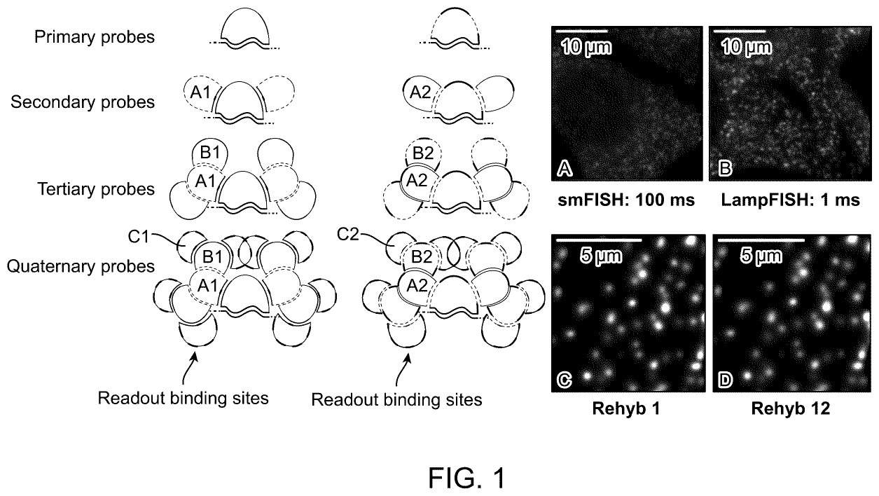

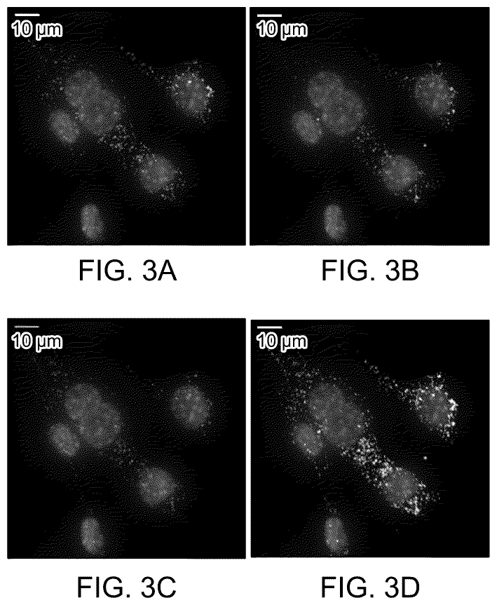

The disclosure herein sets forth an efficient and scalable signal amplification method that can be applied to multiplexed imaging such as RNA and DNA seqFISH as well as immunofluorescence (FIG. 1, FIGS. 3-5). In some embodiments, amplification of tens or hundreds of orthogonal amplicons can be performed at once.

In contrast to other methods such as hybridization chain reaction (HCR), which only allows amplification of several amplicons at once, which is extremely time consuming when imaging tens or hundreds of species from one sample, the primary, secondary, tertiary, quaternary, and readout probes are significantly simpler and less expensive to synthesize than other chemical modifications and are easily compatible with existing enzymatic probe synthesis protocols.

In some embodiments, there is provided a composition for linked-amplifying fluorescence in situ hybridization, comprising a plurality of probes, wherein the composition comprises: one or more primary probes capable of binding one or more targets, wherein each primary probe comprises one or more secondary probe binding sites and optionally one or more readout probe binding sites. In certain embodiments, the composition comprises one or more secondary probes, each capable of binding the primary probe, wherein each secondary probe comprises one or more tertiary probe binding sites or one or more readout probe binding sites. In certain embodiments, the composition optionally comprises one or more tertiary probes, each capable of binding to the secondary probe, wherein each tertiary probe comprises one or more quaternary probe binding sites or one or more readout probe binding sites. In certain embodiments, the composition optionally comprises one or more quaternary probes, each capable of binding to the tertiary probe, wherein each quaternary probe comprises one or more readout probe binding sites. In certain embodiments, the composition comprises one or more readout probes capable of binding to a binding site on the one or more primary, secondary, tertiary, or quaternary probes and capable of being detected. In certain embodiments, the composition comprises one or more molecules capable of stabilizing one or more primary, secondary, tertiary, or quaternary probes when or after the probe is hybridized or after the probe is hybridized.

In some embodiments, there is provided a kit for linked amplification tethered with exponential radiance, comprising a plurality of probes, wherein the composition comprises: wherein each primary probe comprises one or more secondary probe binding sites and optionally one or more readout probe binding sites. In certain embodiments, the composition comprises one or more secondary probes, each capable of binding the primary probe, wherein each secondary probe comprises one or more tertiary probe binding sites or one or more readout probe binding sites. In certain embodiments, the composition optionally comprises one or more tertiary probes, each capable of binding to the secondary probe, wherein each tertiary probe comprises one or more quaternary probe binding sites or one or more readout probe binding sites. In certain embodiments, the composition optionally comprises one or more quaternary probes, each capable of binding to the tertiary probe, wherein each quaternary probe comprises one or more readout probe binding sites. In certain embodiments, the composition comprises one or more readout probes capable of binding to a binding site on the one or more primary, secondary, tertiary, quaternary probes and capable of being detected. In certain embodiments, the kit comprises one or more molecules capable of stabilizing one or more primary, secondary, tertiary, or quaternary probes when or after the probe is hybridized or after the probe is hybridized

In some embodiments, there is provided a method for linked amplification tethered with exponential radiance, comprising a method for linked amplification tethered with exponential radiance, comprising steps of: contacting a sample with one or more primary probes that bind one or more targets, wherein each primary probe hybridizes to the target. In certain embodiments, the method comprises hybridizing one or more secondary probes to the primary probes; wherein each secondary probe comprises one or more tertiary probe binding sites or one or more readout probe binding sites. In certain embodiments, the method optionally comprises hybridizing one or more tertiary probes to at least one secondary probe; and wherein each tertiary probe comprises one or more quaternary probes or one or more readout probe binding sites. In some embodiments, the method optionally comprises hybridizing one or more quaternary probe to at least one tertiary probe, wherein each quaternary probe comprises one or more readout probe binding sites. In some embodiments, the method comprises stabilizing one or more primary, secondary, tertiary, or quaternary probes during or after steps (i)-(iv). In certain embodiments, the method comprises hybridizing readout probes capable of detection to the one or more readout probe binding sites. In certain embodiments, the method comprises imaging the cell so that the interaction of the primary probe to the nucleic acids is detected. In certain embodiments, the method comprises optionally repeating the contacting and imaging steps, each time with a new plurality of detectably labeled readout probes, wherein at least one readout probe for one target differs from at least one other readout probes for the same target in their detectable moieties. In some embodiments, any of the preceding embodiments are repeated either individually or in any combination thereof.

### Samples

In some embodiments, the method comprises analyzing samples, wherein the samples comprise bacterial cells, archaeal cells, eukaryotic cells, or a combination thereof. In certain embodiments, the samples comprise tissues, cells, or extracts from cells. In certain embodiments, the samples comprise biofilms. In certain embodiments, the samples comprise cells obtained from patients.

### Targets

In some embodiments, the targets are selected from transcripts, RNA, DNA loci, chromosomes, DNA, proteins, lipids, glycans, cellular target, organelles, and any combinations thereof. In certain embodiments, the transcripts, RNA, DNA loci, chromosomes, DNA, proteins, lipids, glycans, cellular target, organelles, and any combinations thereof are conjugated to an oligonucleotide.

### Primary, Secondary, Tertiary, and Quaternary Probes

In some embodiments, the primary, secondary, tertiary, or quaternary probe comprises at least one readout probe binding site. In certain embodiments, in any of the previous embodiments, the primary, secondary, tertiary, or quaternary probe comprises at least two readout probe binding sites. In some embodiments, in any of the previous embodiments, the primary, secondary, tertiary, or quaternary probe comprises at least three readout probe binding sites. In some embodiments, in any of the previous embodiments, the primary, secondary, tertiary, or quaternary probe comprises at least four readout probe binding sites. In some embodiments, in any of the previous embodiments, the primary, secondary, tertiary, or quaternary probe comprises at least five readout probe binding sites. In some embodiments, in any of the previous embodiments, the primary, secondary, tertiary, or quaternary probe comprises at least six readout probe binding sites. In some embodiments, in any of the previous embodiments, the primary, secondary, tertiary, or quaternary probe comprises at least seven readout probe binding sites. In some embodiments, in any of the previous embodiments, the primary, secondary, tertiary, or quaternary probe comprises at least eight readout probe binding sites. In some embodiments, in any of the previous embodiments, the primary, secondary, tertiary, or quaternary probe comprises at least nine readout probe binding sites. In some embodiments, in any of the previous embodiments, the primary, secondary, tertiary, or quaternary probe comprises at least 10 readout probe binding sites.

In some embodiments, the primary probe of any of the previous embodiments comprises a nucleic acid sequence complementary to a target nucleic acid sequence.

In some embodiments, the sequence complementary to the target nucleic acid sequence is at least 60%, 65%, 70%, 75%, 80%, 85%, 90%, 91%, 92%, 93%, 94%, 95%, 96%, 97%, 98%, 99%, or 100%. In some embodiments, the sequence complementarity is at least 60%, 65%, 70%, 75%, 80%, 85%, 90%, 91%, 92%, 93%, 94%, 95%, 96%, 97%, 98%, 99%, or 100%.

In some embodiments, the primary probe of any of the preceding embodiments comprises oligonucleotides that are at least 5 nucleotides in length. In some embodiments, the primary probe of any of the preceding embodiments comprises oligonucleotides that are at least 6 nucleotides in length. In some embodiments, the primary probe of any of the preceding embodiments comprises oligonucleotides that are at least 7 nucleotides in length. In some embodiments, the primary probe of any of the preceding embodiments comprises oligonucleotides that are at least 8 nucleotides in length. In some embodiments, the primary probe of any of the preceding embodiments comprises oligonucleotides that are at least 9 nucleotides in length. In some embodiments, the primary probe of any of the preceding embodiments comprises oligonucleotides that are at least 10 nucleotides in length. In some embodiments, the primary probe of any of the preceding embodiments comprises oligonucleotides that are at least 11 nucleotides in length. In some embodiments, the primary probe of any of the preceding embodiments comprises oligonucleotides that are at least 12 nucleotides in length. In some embodiments, the primary probe of any of the preceding embodiments comprises oligonucleotides that are at least 13 nucleotides in length. In some embodiments, the primary probe of any of the preceding embodiments comprises oligonucleotides that are at least 14 nucleotides in length. In some embodiments, the primary probe of any of the preceding embodiments comprises oligonucleotides that are at least 15 nucleotides in length. In some embodiments, the primary probe of any of the preceding embodiments comprises oligonucleotides that are at least 16 nucleotides in length. In some embodiments, the primary probe of any of the preceding embodiments comprises oligonucleotides that are at least 17 nucleotides in length. In some embodiments, the primary probe of any of the preceding embodiments comprises oligonucleotides that are at least 18 nucleotides in length. In some embodiments, the primary probe of any of the preceding embodiments comprises oligonucleotides that are at least 19 nucleotides in length. In some embodiments, the primary probe of any of the preceding embodiments comprises oligonucleotides that are at least 20 nucleotides in length. In some embodiments, the primary probe of any of the preceding embodiments comprises oligonucleotides that are at least 21 nucleotides in length. In some embodiments, the primary probe of any of the preceding embodiments comprises oligonucleotides that are less than 30, 50, 100, 200, 250, 500, 750, or 1000 nucleotides in length.

In some embodiments, the secondary probe of any of the preceding embodiments comprises oligonucleotides that are at least 5 nucleotides in length. In some embodiments, the secondary probe of any of the preceding embodiments comprises oligonucleotides that are at least 6 nucleotides in length. In some embodiments, the secondary probe of any of the preceding embodiments comprises oligonucleotides that are at least 7 nucleotides in length. In some embodiments, the secondary probe of any of the preceding embodiments comprises oligonucleotides that are at least 8 nucleotides in length. In some embodiments, the secondary probe of any of the preceding embodiments comprises oligonucleotides that are at least 9 nucleotides in length. In some embodiments, the secondary probe of any of the preceding embodiments comprises oligonucleotides that are at least 10 nucleotides in length. In some embodiments, the secondary probe of any of the preceding embodiments comprises oligonucleotides that are at least 11 nucleotides in length. In some embodiments, the secondary probe of any of the preceding embodiments comprises oligonucleotides that are at least 12 nucleotides in length. In some embodiments, the secondary probe of any of the preceding embodiments comprises oligonucleotides that are at least 13 nucleotides in length. In some embodiments, the secondary probe of any of the preceding embodiments comprises oligonucleotides that are at least 14 nucleotides in length. In some embodiments, the secondary probe of any of the preceding embodiments comprises oligonucleotides that are at least 15 nucleotides in length. In some embodiments, the secondary probe of any of the preceding embodiments comprises oligonucleotides that are at least 16 nucleotides in length. In some embodiments, the secondary probe of any of the preceding embodiments comprises oligonucleotides that are at least 17 nucleotides in length. In some embodiments, the secondary probe of any of the preceding embodiments comprises oligonucleotides that are at least 18 nucleotides in length. In some embodiments, the secondary probe of any of the preceding embodiments comprises oligonucleotides that are at least 19 nucleotides in length. In some embodiments, the secondary probe of any of the preceding embodiments comprises oligonucleotides that are at least 20 nucleotides in length. In some embodiments, the secondary probe of any of the preceding embodiments comprises oligonucleotides that are at least 21 nucleotides in length. In some embodiments, the secondary probe of any of the preceding embodiments comprises oligonucleotides that are less than 30, 50, 100, 200, 250, 500, 750, or 1000 nucleotides in length.

In some embodiments, the tertiary probe of any of the preceding embodiments comprises oligonucleotides that are at least 5 nucleotides in length. In some embodiments, the tertiary probe of any of the preceding embodiments comprises oligonucleotides that are at least 6 nucleotides in length. In some embodiments, the tertiary probe of any of the preceding embodiments comprises oligonucleotides that are at least 7 nucleotides in length. In some embodiments, the tertiary probe of any of the preceding embodiments comprises oligonucleotides that are at least 8 nucleotides in length. In some embodiments, the tertiary probe of any of the preceding embodiments comprises oligonucleotides that are at least 9 nucleotides in length. In some embodiments, the tertiary probe of any of the preceding embodiments comprises oligonucleotides that are at least 10 nucleotides in length. In some embodiments, the tertiary probe of any of the preceding embodiments comprises oligonucleotides that are at least 10 nucleotides in length In some embodiments, the tertiary probe of any of the preceding embodiments comprises oligonucleotides that are at least 11 nucleotides in length. In some embodiments, the tertiary probe of any of the preceding embodiments comprises oligonucleotides that are at least 12 nucleotides in length. In some embodiments, the tertiary probe of any of the preceding embodiments comprises oligonucleotides that are at least 13 nucleotides in length. In some embodiments, the tertiary probe of any of the preceding embodiments comprises oligonucleotides that are at least 14 nucleotides in length. In some embodiments, the tertiary probe of any of the preceding embodiments comprises oligonucleotides that are at least 15 nucleotides in length. In some embodiments, the tertiary probe of any of the preceding embodiments comprises oligonucleotides that are at least 16 nucleotides in length. In some embodiments, the tertiary probe of any of the preceding embodiments comprises oligonucleotides that are at least 17 nucleotides in length. In some embodiments, the tertiary probe of any of the preceding embodiments comprises oligonucleotides that are at least 18 nucleotides in length. In some embodiments, the tertiary probe of any of the preceding embodiments comprises oligonucleotides that are at least 19 nucleotides in length. In some embodiments, the tertiary probe of any of the preceding embodiments comprises oligonucleotides that are at least 20 nucleotides in length. In some embodiments, the tertiary probe of any of the preceding embodiments comprises oligonucleotides that are at least 21 nucleotides in length. In some embodiments, the tertiary probe of any of the preceding embodiments comprises oligonucleotides that are less than 30, 50, 100, 200, 250, 500, 750, or 1000 nucleotides in length.

In some embodiments, the quaternary probe of any of the preceding embodiments comprises oligonucleotides that are at least 5 nucleotides in length. In some embodiments, the quaternary probe of any of the preceding embodiments comprises oligonucleotides that are at least 6 nucleotides in length. In some embodiments, the quaternary probe of any of the preceding embodiments comprises oligonucleotides that are at least 7 nucleotides in length. In some embodiments, the quaternary probe of any of the preceding embodiments comprises oligonucleotides that are at least 8 nucleotides in length. In some embodiments, the quaternary probe of any of the preceding embodiments comprises oligonucleotides that are at least 9 nucleotides in length. In some embodiments, the quaternary probe of any of the preceding embodiments comprises oligonucleotides that are at least 10 nucleotides in length. In some embodiments, the quaternary probe of any of the preceding embodiments comprises oligonucleotides that are at least 10 nucleotides in length In some embodiments, the quaternary probe of any of the preceding embodiments comprises oligonucleotides that are at least 11 nucleotides in length. In some embodiments, the quaternary probe of any of the preceding embodiments comprises oligonucleotides that are at least 12 nucleotides in length. In some embodiments, the quaternary probe of any of the preceding embodiments comprises oligonucleotides that are at least 13 nucleotides in length. In some embodiments, the quaternary probe of any of the preceding embodiments comprises oligonucleotides that are at least 14 nucleotides in length. In some embodiments, the quaternary probe of any of the preceding embodiments comprises oligonucleotides that are at least 15 nucleotides in length. In some embodiments, the quaternary probe of any of the preceding embodiments comprises oligonucleotides that are at least 16 nucleotides in length. In some embodiments, the quaternary probe of any of the preceding embodiments comprises oligonucleotides that are at least 17 nucleotides in length. In some embodiments, the quaternary probe of any of the preceding embodiments comprises oligonucleotides that are at least 18 nucleotides in length. In some embodiments, the quaternary probe of any of the preceding embodiments comprises oligonucleotides that are at least 19 nucleotides in length. In some embodiments, the quaternary probe of any of the preceding embodiments comprises oligonucleotides that are at least 20 nucleotides in length. In some embodiments, the quaternary probe of any of the preceding embodiments comprises oligonucleotides that are at least 21 nucleotides in length. In some embodiments, the quaternary probe of any of the preceding embodiments comprises oligonucleotides that are less than 30, 50, 100, 200, 250, 500, 750, or 1000 nucleotides in length.

In some embodiments, the secondary probe complements the secondary probe binding site on the primary probe. In some embodiments, the tertiary probe complements the secondary probe binding site on the secondary probe. In some embodiments, the quaternary probe complements the tertiary probe binding site on the tertiary probe. In some embodiments, the probe complements comprise a sequence complementarity that is at least 60%, 65%, 70%, 75%, 80%, 85%, 90%, 91%, 92%, 93%, 94%, 95%, 96%, 97%, 98%, 99%, or 100%.

In some embodiments, the length of the primary, secondary, tertiary, and quaternary probe binding sites range from 5-100 nucleotides. In some embodiments, the length of the primary, secondary, tertiary, and quaternary probe binding sites range from 5-10 nucleotides. In some embodiments, the length of the primary, secondary, tertiary, and quaternary probe binding sites range from 5-20 nucleotides. In some embodiments, the length of the primary, secondary, tertiary, and quaternary probe binding sites range from 5-30 nucleotides. In some embodiments, the length of the primary, secondary, tertiary, and quaternary probe binding sites range from 5-40 nucleotides. In some embodiments, the length of the primary, secondary, tertiary, and quaternary probe binding sites range from 5-50 nucleotides. In some embodiments, the length of the primary, secondary, tertiary, and quaternary probe binding sites range from 5-60 nucleotides. In some embodiments, the length of the primary, secondary, tertiary, and quaternary probe binding sites range from 5-70 nucleotides. In some embodiments, the length of the primary, secondary, tertiary, and quaternary probe binding sites range from 5-80 nucleotides. In some embodiments, the length of the primary, secondary, tertiary, and quaternary probe binding sites range from 5-90 nucleotides.

In some embodiments, the composition or kit of any of the preceding embodiments comprises two or more primary probes capable of binding two or more targets.

In some embodiments, the composition or kit of any of the preceding embodiments comprises two or more secondary probes capable of binding the primary probe, wherein each secondary probe comprises two or more tertiary probe binding sites or two or more readout probe binding sites.

In some embodiments, the composition or kit of any of the preceding embodiments comprises two or more tertiary probes, each capable of binding to two or more tertiary probe binding sites, and wherein each tertiary probe comprises one or more readout probe binding sites.

In some embodiments, the kit of any of the previous embodiments comprises a DNA ligase.

In some embodiments, the methods of any of the previous embodiments comprises contacting a sample with two or more primary probes that bind one or more targets, wherein the two or more primary probes hybridize to the target.

In some embodiments, the methods of any of the preceding embodiments comprises, hybridizing a secondary probe to the ligated primary probe, wherein the secondary probe comprises two or more tertiary probe binding sites or two or more readout probe binding sites.

In some embodiments, the methods of any of the preceding embodiments comprises hybridizing a tertiary probe to at least two secondary probes; and wherein each tertiary probe comprises two or more readout probe binding sites.

In some embodiments, the methods of any of the preceding claims comprises hybridizing readout probes capable of detection to two or more readout-probe binding sites.

In some embodiments, the methods of any of preceding embodiments, further comprises repeating the contacting and imaging steps, each time with a new plurality of detectably labeled readout probes, wherein in each new plurality at least one readout probe for one target differs from at least one readout probe for the same target in a previous plurality, wherein they differ at least in their detectable moieties.

### Amplifier Fragments

In some embodiments, each secondary, each tertiary, and/or each quaternary probe comprises at least two amplifier fragments.

In some embodiments, the secondary, secondary and tertiary, secondary, tertiary, and quaternary, secondary and quaternary, tertiary, tertiary and quaternary, or quaternary probes comprise at least two amplifier fragments.

In certain embodiments, the secondary probe amplifier fragments comprise at least: a first amplifier fragment, wherein the first amplifier fragment comprises a region of complementarity to the primary probe, and wherein the region of complementarity hybridizes to the primary probe. In certain embodiments, the secondary probe amplifier fragments comprise at least: a second amplifier fragment, wherein the second amplifier fragment comprises a region of complementarity to the primary probe, and wherein the region of complementarity hybridizes to the primary probe.

In some embodiments, the tertiary probe amplifier fragments comprise at least: a first amplifier fragment, wherein the first amplifier fragment comprises a region of complementarity to a secondary probe or to the first or second amplifier fragment of the secondary probe, and wherein the region of complementarity hybridizes to the secondary probe or to the first or second amplifier fragment of secondary probe. In certain embodiments, the tertiary probe amplifier fragments comprise at least a second amplifier fragment, wherein the second amplifier fragment comprises a region of complementarity to the secondary probe or to the first or second amplifier fragment of secondary probe, and wherein the region of complementarity hybridizes to the secondary probe or to the first or second amplifier fragment of secondary probe.

In some embodiments, the quaternary probe amplifier fragments comprise at least: a first amplifier fragment, wherein the first amplifier fragment comprises a region of complementarity to a tertiary probe or to the first or second amplifier fragment of the tertiary probe, and wherein the region of complementarity hybridizes to the tertiary probe or to the first or second fragment of tertiary probe. In some embodiments, the quaternary probe amplifier fragments comprise a second fragment, wherein the second fragment comprises a region of complementarity to the tertiary probe or to the first or second amplifier fragment of the tertiary probe, and wherein the region of complementarity hybridizes to the tertiary probe or to the first or second fragment of tertiary probe.

In some embodiments, the composition, kit, or method of any of the previous embodiments comprises a ligase ligates the first or second fragments of any secondary, tertiary, or quaternary amplifier fragments.

In some embodiments, the amplifier fragments of any of the preceding embodiments comprises oligonucleotides that are at least 5 nucleotides in length. In some embodiments, the amplifier fragments of any of the preceding embodiments comprises oligonucleotides that are at least 6 nucleotides in length. In some embodiments, the amplifier fragments of any of the preceding embodiments comprises oligonucleotides that are at least 7 nucleotides in length. In some embodiments, the amplifier fragments of any of the preceding embodiments comprises oligonucleotides that are at least 8 nucleotides in length. In some embodiments, the amplifier fragments of any of the preceding embodiments comprises oligonucleotides that are at least 9 nucleotides in length. In some embodiments, the amplifier fragments of any of the preceding embodiments comprises oligonucleotides that are at least 10 nucleotides in length. In some embodiments, the amplifier fragments of any of the preceding embodiments comprises oligonucleotides that are at least 11 nucleotides in length. In some embodiments, the amplifier fragments of any of the preceding embodiments comprises oligonucleotides that are at least 12 nucleotides in length. In some embodiments, the amplifier fragments of any of the preceding embodiments comprises oligonucleotides that are at least 13 nucleotides in length. In some embodiments, the amplifier fragments of any of the preceding embodiments comprises oligonucleotides that are at least 14 nucleotides in length. In some embodiments, the amplifier fragments of any of the preceding embodiments comprises oligonucleotides that are at least 15 nucleotides in length. In some embodiments, the amplifier fragments of any of the preceding embodiments comprises oligonucleotides that are at least 16 nucleotides in length. In some embodiments, the amplifier fragments of any of the preceding embodiments comprises oligonucleotides that are at least 17 nucleotides in length. In some embodiments, the amplifier fragments of any of the preceding embodiments comprises oligonucleotides that are at least 18 nucleotides in length. In some embodiments, the amplifier fragments of any of the preceding embodiments comprises oligonucleotides that are at least 19 nucleotides in length. In some embodiments, the amplifier fragments of any of the preceding embodiments comprises oligonucleotides that are at least 20 nucleotides in length. In some embodiments, the amplifier fragments of any of the preceding embodiments comprises oligonucleotides that are at least 21 nucleotides in length. In some embodiments, the amplifier fragments of any of the preceding embodiments comprises oligonucleotides that are less than 30, 50, 100, 200, 250, 500, 750, or 1000 nucleotides in length.

In some embodiments, the region of complementarity of any of the previous embodiments comprises a region of the oligonucleotides of any of the previous embodiments that is at least 5 nucleotides in length. In some embodiments, the region of complementarity of any of the previous embodiments comprises a region of the oligonucleotides of any of the previous embodiments that is at least 6 nucleotides in length. In some embodiments, the region of complementarity of any of the previous embodiments comprises a region of the oligonucleotides of any of the previous embodiments that is at least 7 nucleotides in length. In some embodiments, the region of complementarity of any of the previous embodiments comprises a region of the oligonucleotides of any of the previous embodiments that is at least 8 nucleotides in length. In some embodiments, the region of complementarity of any of the previous embodiments comprises a region of the oligonucleotides of any of the previous embodiments that is at least 9 nucleotides in length. In some embodiments, the region of complementarity of any of the previous embodiments comprises a region of the oligonucleotides of any of the previous embodiments that is at least 10 nucleotides in length. In some embodiments, the region of complementarity of any of the previous embodiments, comprises a region of the oligonucleotides of any of the previous embodiments that is at least 11 nucleotides in length. In some embodiments, the region of complementarity of any of the previous embodiments comprises a region of the oligonucleotides of any of the previous embodiments that is at least 12 nucleotides in length. In some embodiments, the region of complementarity of any of the previous embodiments comprises a region of the oligonucleotides of any of the previous embodiments that is at least 13 nucleotides in length. In some embodiments, the region of complementarity of any of the previous embodiments comprises a region of the oligonucleotides of any of the previous embodiments that is at least 14 nucleotides in length. In some embodiments, the region of complementarity of any of the previous embodiments comprises a region of the oligonucleotides of any of the previous embodiments that is at least 15 nucleotides in length. In some embodiments, the region of complementarity of any of the previous embodiments comprises a region of the oligonucleotides of any of the previous embodiments that is at least 16 nucleotides in length. In some embodiments, the region of complementarity of any of the previous embodiments comprises a region of the oligonucleotides of any of the previous embodiments that is at least 17 nucleotides in length. In some embodiments, the region of complementarity of any of the previous embodiments comprises a region of the oligonucleotides of any of the previous embodiments that is at least 18 nucleotides in length. In some embodiments, the region of complementarity of any of the previous embodiments comprises a region of the oligonucleotides of any of the previous embodiments that is at least 19 nucleotides in length. In some embodiments, the region of complementarity of any of the previous embodiments comprises a region of the oligonucleotides of any of the previous embodiments that is at least 20 nucleotides in length. In some embodiments, the region of complementarity of any of the previous embodiments comprises a region of the oligonucleotides of any of the previous embodiments that is at least 21 nucleotides in length. In some embodiments, the region of complementarity of any of the previous embodiments comprises a region of the oligonucleotides of any of the previous embodiments that are less than 30, 50, 100, 200, 250, 500, 750, or 1000 nucleotides in length.

In some embodiments, the region of complementarity of any of the previous embodiments comprises a sequence complementarity that is at least 60%, 65%, 70%, 75%, 80%, 85%, 90%, 91%, 92%, 93%, 94%, 95%, 96%, 97%, 98%, 99%, or 100%.

### Splint Sequence

In some embodiments, the composition, kit, or method of any of the embodiments comprises a splint sequence.

In some embodiments, each splint sequence of any of the preceding embodiments comprises an oligonucleotide that is at least 5 nucleotides in length. In some embodiments, the splint sequence of any of the preceding embodiments comprises an oligonucleotide that isat least 6 nucleotides in length. In some embodiments, the splint sequence of any of the preceding embodiments comprises an oligonucleotide that isat least 7 nucleotides in length. In some embodiments, the splint sequence of any of the preceding embodiments comprises an oligonucleotide that isat least 8 nucleotides in length. In some embodiments, the splint sequence of any of the preceding embodiments comprises an oligonucleotide that isat least 9 nucleotides in length. In some embodiments, the splint sequence of any of the preceding embodiments comprises an oligonucleotide that isat least 10 nucleotides in length. In some embodiments, the splint sequence of any of the preceding embodiments comprises an oligonucleotide that isat least 10 nucleotides in length In some embodiments, the splint sequence of any of the preceding embodiments comprises an oligonucleotide that isat least 11 nucleotides in length. In some embodiments, the splint sequence of any of the preceding embodiments comprises an oligonucleotide that isat least 12 nucleotides in length. In some embodiments, the splint sequence of any of the preceding embodiments comprises an oligonucleotide that isat least 13 nucleotides in length. In some embodiments, the splint sequence of any of the preceding embodiments comprises an oligonucleotide that isat least 14 nucleotides in length. In some embodiments, the splint sequence of any of the preceding embodiments comprises an oligonucleotide that isat least 15 nucleotides in length. In some embodiments, the splint sequence of any of the preceding embodiments comprises an oligonucleotide that isat least 16 nucleotides in length. In some embodiments, the splint sequence of any of the preceding embodiments comprises an oligonucleotide that isat least 17 nucleotides in length. In some embodiments, the splint sequence of any of the preceding embodiments comprises an oligonucleotide that isat least 18 nucleotides in length. In some embodiments, the splint sequence of any of the preceding embodiments comprises an oligonucleotide that isat least 19 nucleotides in length. In some embodiments, the splint sequence of any of the preceding embodiments comprises an oligonucleotide that isat least 20 nucleotides in length. In some embodiments, the splint sequence of any of the preceding embodiments comprises an oligonucleotide that isat least 21 nucleotides in length. In some embodiments, the splint sequence of any of the preceding embodiments comprises an oligonucleotide that is less than 30, 50, 100, 200, 250, 500, 750, or 1000 nucleotides in length.

In some embodiments, the splint sequence hybridizes to the first amplifier fragment, the second amplifier fragment, or both amplifier fragments of the secondary, tertiary, or quaternary amplifier fragments.

In certain embodiments, the splint sequence comprises at least two splint sequence fragments.

In some embodiments, the splint sequence fragments of any of the preceding embodiments comprises an oligonucleotide that isat least 5 nucleotides in length. In some embodiments, the splint sequence fragments of any of the preceding embodiments comprises an oligonucleotide that isat least 6 nucleotides in length. In some embodiments, the splint sequence fragments of any of the preceding embodiments comprises an oligonucleotide that isat least 7 nucleotides in length. In some embodiments, the splint sequence fragments of any of the preceding embodiments comprises an oligonucleotide that isat least 8 nucleotides in length. In some embodiments, the splint sequence fragments of any of the preceding embodiments comprises an oligonucleotide that isat least 9 nucleotides in length. In some embodiments, the splint sequence fragments of any of the preceding embodiments comprises an oligonucleotide that isat least 10 nucleotides in length. In some embodiments, the splint sequence fragments of any of the preceding embodiments comprises an oligonucleotide that isat least 10 nucleotides in length In some embodiments, the splint sequence fragments of any of the preceding embodiments comprises an oligonucleotide that isat least 11 nucleotides in length. In some embodiments, the splint sequence fragments of any of the preceding embodiments comprises an oligonucleotide that isat least 12 nucleotides in length. In some embodiments, the splint sequence fragments of any of the preceding embodiments comprises an oligonucleotide that isat least 13 nucleotides in length. In some embodiments, the splint sequence fragments of any of the preceding embodiments comprises an oligonucleotide that isat least 14 nucleotides in length. In some embodiments, the splint sequence fragments of any of the preceding embodiments comprises an oligonucleotide that isat least 15 nucleotides in length. In some embodiments, the splint sequence fragments of any of the preceding embodiments comprises an oligonucleotide that isat least 16 nucleotides in length. In some embodiments, the splint sequence fragments of any of the preceding embodiments comprises an oligonucleotide that isat least 17 nucleotides in length. In some embodiments, the splint sequence fragments of any of the preceding embodiments comprises an oligonucleotide that isat least 18 nucleotides in length. In some embodiments, the splint sequence fragments of any of the preceding embodiments comprises an oligonucleotide that isat least 19 nucleotides in length. In some embodiments, the splint sequence fragments of any of the preceding embodiments comprises an oligonucleotide that isat least 20 nucleotides in length. In some embodiments, the splint sequence fragments of any of the preceding embodiments comprises an oligonucleotide that isat least 21 nucleotides in length. In some embodiments, the splint sequence fragments of any of the preceding embodiments comprises an oligonucleotide that is less than 30, 50, 100, 200, 250, 500, 750, or 1000 nucleotides in length.

In some embodiments, the splint sequence fragments are ligated.

### Stabilization of Probes

In some embodiments, the method comprises stabilizing the primary, secondary, tertiary probes, or quaternary probes. In some embodiments, the method comprises stabilizing the primary probe. In some embodiments, the method comprises stabilizing the secondary probe. In some embodiments, the method comprises stabilizing the tertiary probe. In some embodiments, the method further comprises stabilizing the quaternary probe.

In some embodiments, the probes are stabilized by methods selected from enzyme ligation, chemical ligation, UV crosslinking with or without oligo splint probes, hybridization of splint probes, crosslinking through a matrix, and chemical crosslinking, or any combination thereof. In certain embodiments, the enzymes used for enzyme ligation are selected from T4 Ligase, T7 Ligase, quick ligase, T3 ligase, and ampligase. In certain embodiments, the chemical ligation is selected from comprise amine-phosphate, diamine, and thiol ligation. In certain embodiments, the crosslinking through the matrix comprises a hydrogel made from polyacrylamide or agarose. In certain embodiments, the chemical crosslinking for stabilization is selected from paraformaldehyde, glutaraldehyde, and reversible crosslinkers such as DSP (dithiobis succinimidyl propionate). In certain embodiments, the splint probes comprise locked nucleic acid (LNA) or peptide nucleic acid (PNA).

In some embodiments, the primary, secondary, tertiary, or quaternary probes of any of the previous embodiments are cis-ligated through one or more additional molecules, such as an oligonucleotide probe, LNA or PNA, or a protein, molecular complexes, or small chemical molecules.

In some embodiments, the primary, secondary, tertiary, or quaternary probes of any of the previous embodiments are UV cis-ligated either directly or indirectly through intermediate molecules.

In some embodiments, the primary, secondary, tertiary, or quaternary probes of any of the previous embodiments are cis-ligated with the cellular or sample matrix directly or through intermediate molecules. In certain embodiments, the primary, secondary, tertiary, or quaternary probes of any of the previous embodiments are cis-ligated using chemical crosslinkers comprising paraformaldehyde, glutaraldehyde, or reversible crosslinkers. In certain embodiments, the primary, secondary, tertiary, or quaternary probes of any of the previous embodiments are cis-ligated using a matrix comprising a native tissue matrix, tissue matrix, or an exogenous matrix. In certain embodiments, the exogenous matrix comprises a hydrogel.

In some embodiments, the primary, secondary, tertiary, or quaternary probes of any of the previous embodiments are stabilized before a subsequent round of probe hybridization. In certain embodiments, the primary, secondary, tertiary, or quaternary probes of any of the previous embodiments are stabilized before a stripping step.

In some embodiments, the primary, secondary, tertiary, or quaternary probes of any of the previous embodiments are ligated by use of a ligase. In certain embodiments, the probes are ligated either cis or trans. In some embodiments, the primary, secondary, tertiary, or quaternary probes of any of the previous embodiments are cis-ligated. In some embodiments, the primary, secondary, tertiary, or quaternary probes of any of the previous embodiments are trans-ligated.

In some embodiments, the primary, secondary, tertiary, or quaternary probes of any of the previous embodiments are cis-ligated by acrylamide polymerization.

In some embodiments, the primary, secondary, tertiary, or quaternary probes of any of the previous embodiments are cis-ligated by click chemistry.

In some embodiments, the primary, secondary, tertiary, or quaternary probes of any of the previous embodiments are cis-ligated by reactive groups, wherein the reactive groups form a reactive pair selected from alkenes, alkynes, azides, amides, amine, nitrones, phosphates, tetrazines, and tetrazoles.

In some embodiments, the methods of any of the preceding embodiments use a primary probe that is ligated or cross-linked. In some embodiments, the methods of any of the preceding embodiments comprises a primary probe that is ligated or cross-linked either cis or trans.

In some embodiments, the primary, secondary, or tertiary probes of any of the preceding embodiments are ligated or cross-linked.

In some embodiments, the primary, secondary, or tertiary probes of any of the preceding embodiments are cis-ligated.

In some embodiments, the enzyme of any of the previous embodiments comprises a DNA or RNA ligase, or DNA polymerase or RNA polymerase, and or combination of any above.

In some embodiments, the kit of any of the previous embodiments comprises a DNA ligase.

### Fluorophores

In some embodiments, the composition, kit, or method of any of the embodiments comprises a detectable moiety. In some embodiments, the composition, kit, or method of any one of the preceding embodiments comprises at least two different detectable moieties. In certain embodiments, the detectable moieties are the same.

In some embodiments, the detectable moiety is any fluorophore deemed suitable by those of skill in the arts.

In some embodiments, the detectable moieties include but are not limited to fluorescein, rhodamine, Alexa Fluors, DyLight fluors, ATTO Dyes, or any analogs or derivatives thereof. In certain embodiments, the detectable moieties include but are not limited to fluorescein and chemical derivatives of fluorescein; Eosin; Carboxyfluorescein; Fluorescein isothiocyanate (FITC); Fluorescein amidite (FAM); Erythrosine; Rose Bengal; fluorescein secreted from the bacterium Pseudomonas aeruginosa; Methylene blue; Laser dyes; Rhodamine dyes (e.g., Rhodamine. Rhodamine 6G, Rhodamine B, Rhodamine 123, Auramine O, Sulforhodamine 101, Sulforhodamine B, and Texas Red).

In some embodiments, the detectable moieties include but are not limited to ATTO dyes; Acridine dyes (e.g., Acridine orange. Acridine yellow); Alexa Fluor; 7-Amino actinomycin D; 8-Anilinonaphthalene-1-sulfonate; Auramine-rhodamine stain; Benzanthrone; 5,12-Bis(phenylethynyl) naphthacene; 9,10-Bis(phenylethynyl)anthracene; Blacklight paint; Brainbow; Calcein; Carboxyfluorescein; Carboxyfluorescein diacetate succinimidyl ester; Carboxyfluorescein succinimidyl ester; 1-Chloro-9,10-bis(phenylethynyl)anthracene; 2-Chloro-9,10-bis(phenylethynyl)anthracene; 2-Chloro-9,10-diphenylanthracene; Coumarin; Cyanine dyes (e.g., Cyanine such as Cy3 and Cy5, DiOC6, SYBR Green I); DAPI, Dark quencher, DyLight Fluor, Fluo-4, FluoProbes; Fluorone dyes (e.g., Calcein, Carboxyfluorescein, Carboxyfluorescein diacetate succinimidyl ester, Carboxyfluorescein succinimidyl ester, Eosin, Eosin B, Eosin Y, Erythrosine, Fluorescein, Fluorescein isothiocyanate, Fluorescein amidite, Indian yellow, Merbromin); Fluoro-Jade stain; Fura-2; Fura-2-acetoxymethyl ester; Green fluorescent protein, Hoechst stain, Indian yellow, Indo-1, Lucifer yellow, Luciferin, Merocyanine, Optical brightener, Oxazin dyes (e.g., Cresyl violet, Nile blue, Nile red); Perylene; Phenanthridine dyes (Ethidium bromide and Propidium iodide); Phloxine, Phycobilin, Phycoerythrin, Phycoerythrobilin, Pyranine, Rhodamine, Rhodamine 123, Rhodamine 6G, RiboGreen, RoGFP, Rubrene, SYBR Green I, (E)-Stilbene, (Z)-Stilbene, Sulforhodamine 101, Sulforhodamine B, Synapto-pHluorin, Tetraphenyl butadiene. Tetrasodium tris(bathophenanthroline disulfonate) ruthenium(II). Texas Red, TSQ, Umbelliferone, or Yellow fluorescent protein.

In some embodiments, the detectable moieties include but are not limited to Alexa Fluor family of fluorescent dyes (Molecular Probes. Oregon). Alexa Fluor dyes are widely used as cell and tissue labels in fluorescence microscopy and cell biology. The excitation and emission spectra of the Alexa Fluor series cover the visible spectrum and extend into the infrared. The individual members of the family are numbered according roughly to their excitation maxima (in nm). Certain Alexa Fluor dyes are synthesized through sulfonation of coumarin, rhodamine, xanthene (such as fluorescein), and cyanine dyes. In some embodiments, sulfonation makes Alexa Fluor dyes negatively charged and hydrophilic. In some embodiments, Alexa Fluor dyes are more stable, brighter, and less pH-sensitive than common dyes (e.g. fluorescein, rhodamine) of comparable excitation and emission, and to some extent the newer cyanine series. Exemplary Alexa Fluor dyes include but are not limited to Alexa-350, Alexa-405, Alexa-430, Alexa-488, Alexa-500, Alexa-514, Alexa-532, Alexa-546, Alexa-555, Alexa-568, Alexa-594, Alexa-610, Alexa-633, Alexa-647, Alexa-660, Alexa-680, Alexa-700, or Alexa-750.

In some embodiments, the detectable moieties comprise one or more of the DyLight Fluor family of fluorescent dyes (Dyomics and Thermo Fisher Scientific). Exemplary DyLight Fluor family dyes include but are not limited to DyLight-350, DyLight-405, DyLight-488, DyLight-549, DyLight-594, DyLight-633, DyLight-649, DyLight-680, DyLight-750, or DyLight-800.

In some embodiments, the detectable moieties comprises a nanomaterial. In some embodiments, the fluorophore is a nanoparticle. In some embodiments, the detectable moiety is or comprises a quantum dot. In some embodiments, the fluorophore is a quantum dot. In some embodiments, the detectable moiety comprises a quantum dot. In some embodiments, the detectable moiety is or comprises a gold nanoparticle. In some embodiments, the detectable moiety is a gold nanoparticle. In some embodiments, the detectable moiety comprises a gold nanoparticle.

### Readout Probe

In some embodiments, the one or more readout probes of any of the previous embodiments comprise an oligonucleotide or antibody with a detectable moiety.

In some embodiments, the one or more readout probes of any of the preceding embodiments comprise oligonucleotides with the same sequence.

In some embodiments, the one or more readout probes of any of the preceding embodiments comprise oligonucleotides with different sequences.

In some embodiments, the one or more readout probes of any of the preceding embodiments comprise oligonucleotides that are at least 17 nucleotides in length.

In some embodiments, the readout probe of any of the preceding embodiments comprises oligonucleotides that are at least 5 nucleotides in length. In some embodiments, the readout probe of any of the preceding embodiments comprises oligonucleotides that are at least 10 nucleotides in length In some embodiments, the readout probe of any of the preceding embodiments comprises oligonucleotides that are at least 11 nucleotides in length. In some embodiments, the readout probe of any of the preceding embodiments comprises oligonucleotides that are at least 12 nucleotides in length. In some embodiments, the readout probe of any of the preceding embodiments comprises oligonucleotides that are at least 13 nucleotides in length. In some embodiments, the readout probe of any of the preceding embodiments comprises oligonucleotides that are at least 14 nucleotides in length. In some embodiments, the readout probe of any of the preceding embodiments comprises oligonucleotides that are at least 15 nucleotides in length. In some embodiments, the readout probe of any of the preceding embodiments comprises oligonucleotides that are at least 16 nucleotides in length. In some embodiments, the readout probe of any of the preceding embodiments comprises oligonucleotides that are at least 17 nucleotides in length. In some embodiments, the readout probe of any of the preceding embodiments comprises oligonucleotides that are at least 18 nucleotides in length. In some embodiments, the readout probe of any of the preceding embodiments comprises oligonucleotides that are at least 19 nucleotides in length. In some embodiments, the readout probe of any of the preceding embodiments comprises oligonucleotides that are at least 20 nucleotides in length. In some embodiments, the readout probe of any of the preceding embodiments comprises oligonucleotides that are at least 21 nucleotides in length. In some embodiments, the readout probe of any of the preceding embodiments comprises oligonucleotides that are less than 30, 50, 100, 200, 250, 500, 750, or 1000 nucleotides in length.

In some embodiments, the readout probe complements the readout probe binding site on the primary probe. In some embodiments, the readout probe complements the readout probe binding site on the secondary probe. In some embodiments, the readout probe complements the readout probe binding site on the tertiary probe. In some embodiments, the readout probe complements the readout probe binding site on the quaternary probe. In some embodiments, the readout probe complements to a splint sequence fragment. In some embodiments, the probe complements comprise a sequence complementarity that is at least 60%, 65%, 70%, 75%, 80%, 85%, 90%, 91%, 92%, 93%, 94%, 95%, 96%, 97%, 98%, 99%, or 100%.

In some embodiments, the length of the readout probe binding sites range from 5-100 nucleotides. In some embodiments, the length of the readout probe binding sites range from 5-10 nucleotides. In some embodiments, the length of the readout probe binding sites range from 5-20 nucleotides. In some embodiments, the length of the readout probe binding sites range from 5-30 nucleotides. In some embodiments, the length of the readout probe binding sites range from 5-40 nucleotides. In some embodiments, the length of the readout probe binding sites range from 5-50 nucleotides. In some embodiments, the length of the readout probe binding sites range from 5-60 nucleotides. In some embodiments, the length of the readout probe binding sites range from 5-70 nucleotides. In some embodiments, the length of the readout probe binding sites range from 5-80 nucleotides. In some embodiments, the length of the readout probe binding sites range from 5-90 nucleotides.

In some embodiments, composition or kit of any of the previous embodiments comprises two or more readout probes capable of binding to at least one of the one or more readout probe binding site and capable of being detected.

In some embodiments, composition or kit of any of the previous embodiments comprises two or more readout probes capable of binding to at least one of the one or more readout probe binding site and capable of being detected.

### Imaging the Sample

In some embodiments, the method comprises imaging the probes or barcodes. In some embodiments, the method comprises imaging the target probes or barcodes. As understood by a person having ordinary skill in the art, different technologies can be used for the imaging steps.

In some embodiments, the imaging methods comprise but are not limited to epi-fluorescence microscopy, confocal microscopy, the different types of super-resolution microscopy (PALM/STORM, SSIM/GSD/STED), and light sheet microscopy (SPIM and etc).

In some embodiments, the imaging methods comprise exemplary super resolution technologies include, but are not limited to 15M and 4Pi-microscopy, Stimulated Emission Depletion microscopy (STEDM), Ground State Depletion microscopy (GSDM), Spatially Structured Illumination microscopy (SSIM), Photo-Activated Localization Microscopy (PALM), Reversible Saturable Optically Linear Fluorescent Transition (RESOLFT), Total Internal Reflection Fluorescence Microscope (TIRFM), Fluorescence-PALM (FPALM), Stochastical Optical Reconstruction Microscopy (STORM), Fluorescence Imaging with One-Nanometer Accuracy (FIONA), and combinations thereof. For examples: Chi, 2009 “Super-resolution microscopy: breaking the limits,” Nature Methods 6(1): 15-18; Blow 2008, “New ways to see a smaller world,” Nature 456:825-828; Hell, et al, 2007, “Far-Field Optical Nanoscopy,” Science 316: 1153; R. Heintzmann and G. Ficz, 2006, “Breaking the resolution limit in light microscopy,” Briefings in Functional Genomics and Proteomics 5(4):289-301; Garini et al., 2005, “From micro to nano: recent advances in high-resolution microscopy,” Current Opinion in Biotechnology 16:3-12; and Bewersdorf et al, 2006, “Comparison of I'M and 4Pi-microscopy,” 222(2): 105-1 17; and Wells, 2004, “Man the Nanoscopes,” JCB 164(3):337-340.

In some embodiments, electron microscopes (EM) are used for imaging.

In some embodiments, an imaging step detects a target. In some embodiments, an imaging step localizes a target. In some embodiments, an imaging step provides three-dimensional spatial information of a target. In some embodiments, an imaging step quantifies a target. By using multiple contacting and imaging steps, provided methods are capable of providing spatial and/or quantitative information for a large number of targets in surprisingly high throughput. For example, when using F detectably different types of labels, spatial and/or quantitative information of up to F_{N} targets can be obtained after N contacting and imaging steps.

Certain techniques for imaging are known in the art. See, for example, International PCT Patent Application No. PCT/US2014/036258, filed Apr. 30, 2014 and titled MULTIPLEX LABELING OF MOLECULES BY SEQUENTIAL HYBRIDIZATION BARCODING, the entire contents of which are herein incorporated by reference in its entirety for all purposes.

In some embodiments, the method comprises analyzing cell size and shape, markers, immunofluorescence measurements, or any combinations thereof.

### Removing Probes

In some embodiments, the method of any of the preceding embodiments, comprises washing the sample after each step. In certain embodiments, the sample is washed with a buffer that removes non-specific hybridization reactions. In certain embodiments, formamide is used in the wash step. In certain embodiments, the wash buffer is stringent. In certain embodiments, the wash buffer comprises 10% formamide, 2×SSC, and 0.1% triton X-100s.

In some embodiments, the method comprises a step of removing the one or more probes after one or more imaging steps. In some embodiments, the step of removing the probes comprises contacting the plurality of readout probes with an enzyme that digests the probes. In some embodiments, the step of removing comprises contacting the plurality of probes with a DNase, contacting the plurality of probes with an RNase, photobleaching, strand displacement, formamide wash, heat denaturation, chemical denaturation, cleavage, or combinations thereof. In some embodiments, the step of removing comprises photobleaching to remove the probes.

In some embodiments, the method further comprises comprising removing the readout probes after one or more imaging steps. In some embodiments, the method comprises the step of removing comprises contacting the plurality of readout probes with an enzyme that digests a readout probe. In some embodiments, the method comprises removing the readout probes by using stripping reagents, wash buffers, photobleaching, chemical bleaching, and any combinations thereof. In some embodiments, the method comprises contacting the plurality of target readout probes with a DNase, contacting the plurality of target probes with an RNase, photobleaching, strand displacement, formamide wash, heat denaturation, or combinations thereof. In some embodiments, the target readout probes are removed by photobleaching.

In some embodiments, the method comprises clearing the sample. In some embodiments the sample is cleared by CLARITY. In some embodiments, the sample is cleared following hydrogel embedding.

Certain techniques for removing probes are known in the art. See, for example, International PCT Patent Application No. PCT/US2014/036258, filed Apr. 30, 2014 and titled MULTIPLEX LABELING OF MOLECULES BY SEQUENTIAL HYBRIDIZATION BARCODING, the entire contents of which are herein incorporated by reference in its entirety for all purposes.

The following non-limiting methods are provided to further illustrate the embodiments of the invention disclosed herein. It should be appreciated by those of skill in the art that the techniques disclosed in the examples that follow represent approaches that have been found to function well in the practice of several embodiments of the invention, and thus be considered to constitute examples of modes for its practice. However, those of skill in the art should, in light of the present disclosure, appreciate that many changes can be made in the specific embodiments that are disclosed and still obtain a like or similar result without departing from the spirit and the scope of the invention.

### METHODS

### Probe Design and Synthesis

Gene- or antibody barcode-specific primary probes are designed to target an RNA or DNA sequence of interest as is common in the art of FISH. Commonly, 25-35 nucleotide complementary regions are used. For RNA FISH, 10-30 nucleotides of these are designed for each transcript.

Instead of direct readout probe sites, the primary probes bear 1-4 sites for the first round of amplification padlock probes (hereafter referred to as the ‘A’ sequence), as well as 5′ and 3′ common sequence that can be used to hybridize a splint for ligation

Three pools of 5′-phosphorylated amplification probes are synthesized. The first pool (Secondary probes) binds to the ‘A’ sequence on the primary probe and displays multiple repeats of the ‘B’ sequence. The second pool (Tertiary probes) binds to the ‘B’ sequence and displays multiple repeats of the ‘A’ sequence. The third pool (Readout probes) binds to the ‘A’ sequence or the ‘B’ sequence and displays binding sites for fluorescently-conjugated readout probes.

### Amplification Protocol

An optional pre-clearing of the sample, such as by 8% SDS or Triton X-100 at RT for 30 minutes, or ethanol or methanol at −20° ° C. for 1-24 hr may be performed.

The primary probes or antibodies are incubated with the sample as normal for smFISH, for example in a standard hybridization, or a standard immunofluorescence blocking buffer

If the primary probes are 5′-phosphorylated, they are ligated by a ligase.

If the primary probes have a 5′ acrydite group, the sample is embedded in a polyacrylamide gel. After polymerization, digestion/clearing of the sample is performed, for example with proteinase K in 1% SDS/50 mM Tris HCl/2 mM CaCl₂) for 1-24 hr at 37° C.

The sample is repeated hybridized, washed, stabilized, and linked and washed with secondary and tertiary probes, and then readout with readout oligos.

The amplified sample may be optionally postfixed. This step may ensure the stability of the amplification over many imaging rounds of formamide stripping and rehybridization.

### Imaging

Chemically-conjugated fluorescent readout probes are then hybridized either directly or using short bridges to the unique 10-17 nucleotide sequences on each readout adapter at RT −37° C. in a suitable buffer, for example 10% ethylene carbonate, 4×SSC and 10% 6.5-10 kDa dextran sulfate, for 5-40 min followed by a mild wash such as 10% formamide, 2×SSC and 0.1% triton X-100, then a nuclear stain such as DAPI.

Imaging is carried out as in normal smFISH in an anti-bleaching buffer system commonly consisting of 4×SSC, 25 mM Tris HCl, glucose oxidase, catalase, and Trolox, using appropriate filter sets and laser illumination. Commonly laser powers of 50-500 mW and objective lenses between 20× and 100× are used on a confocal or widefield microscope equipped with an sCMOS camera. Importantly, amplification allows significantly shorter exposure times per Z slice, down to 10 ms on a spinning disk confocal microscope, which significantly reduces the background signal. Alternatively, a widefield setup may be used to image smFISH in tissue samples, which normally require confocal imaging.

Fluorescent readout probes are stripped by 60% or less formamide wash with or without strand displacement using a 5-10 nucleotide toehold. Strand displacement may help fully extinguish fluorescent signal from higher-GC content readout sequences.

### Experiment 1

The methods disclosed herein target either nucleic acids or proteins of interest in situ or in vitro using DNA primary probes or DNA-conjugated antibodies, which can be either ligated into a circular single stranded DNA (ssDNA) or crosslinked into a polyacrylamide gel and contain secondary probe binding sites.

As an example, the primary binding sites (FIG. 1) are hybridized with ssDNA primary probes, which could contain locked nucleic acid (LNA) or other modified oligonucleotides for higher affinity.

The primary probe is modified at the 5′ end by either phosphate, which allows covalent circularization by DNA ligase, or by acrylamide, which allows free radical polymerization into a polyacrylamide cross-link. This ligation/polymerization step stabilizes the primary probe binding to the targeted nucleotides during subsequent rounds of hybridization and washing.

Using an enzyme to ligate probes has several advantages over designing probes to ligate using click chemistry. Enzymatic ligation allows greater specificity because the two ends of the probe must be right next to each other in order to ligate. In contrast click chemistry often will ligate the ends of non-adjacent probes. The use of enzymatic ligation ensure the amplication is very specific. Still further, the use of enzymes allows the inexpensive production of oligonucleotides in massive quantities for experiments. An individual probes that utilize click chemistry are often purchased at roughly 1000 dollars U.S. per probe.

Next, padlock-style secondary probes with 5′-phosphorylation modification are hybridized to the one or several secondary binding site(s) within the primary probes, and then ligated with DNA ligase. These secondary probes contain two or more tertiary probe binding sites. After the hybridization and ligation of the secondary probes, padlock-style tertiary probes with 5′-phosphorylation modification are hybridized to the two or more tertiary binding sites within the secondary probes, and then ligated with DNA ligase. These tertiary probes contain two or more secondary probe binding sites.

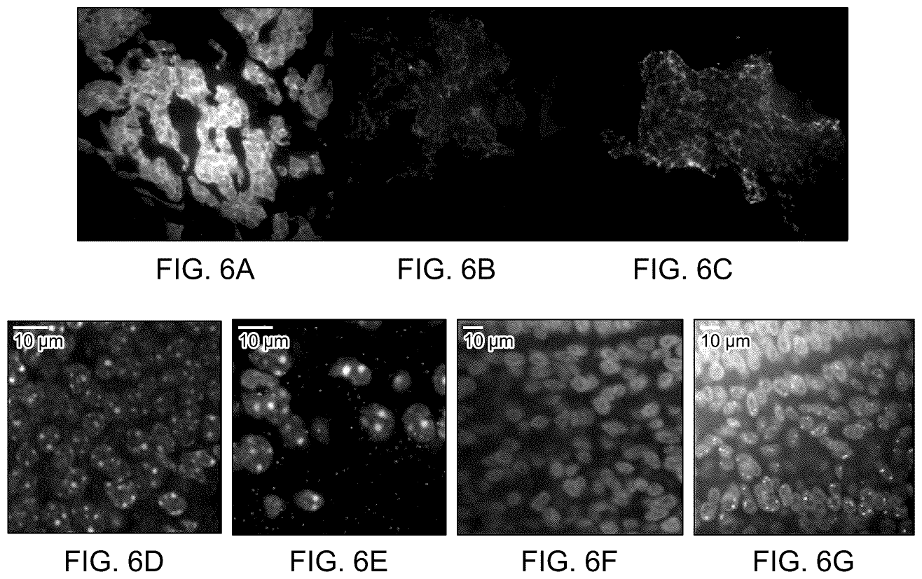

Importantly, the above steps can be iterated. Because secondary and tertiary probes carry two or more secondary or tertiary probe binding sites, the iteration of the steps allows exponential amplification of the secondary and tertiary probes. For example, two rounds of secondary and tertiary probe hybridization and ligation respectively with two probe binding sites each can amplify one secondary probe binding sites to 24=16 of secondary probe binding sites. Additional steps of secondary and tertiary probe hybridization and ligation can increase the number of readout probe binding sites theoretically thousands of times; in practice, several several hundred times was achieved (FIG. 1 and FIG. 6E).

After a desired number of iterations, padlock-style probes with 5′ end phosphorylation modification, which contain multiple readout probe binding sites, are similarly hybridized and ligated. The readout binding sites are then visualized by 17-nt or shorter readout probes that are conjugated to fluorophores, which provides exponentially amplified signals compared to those directly from primary probes. The fluorescent signals from the readout probes can be stripped off by using 60% or lower formamide solution without affecting the primary probe and the padlock structures (FIG. 1).

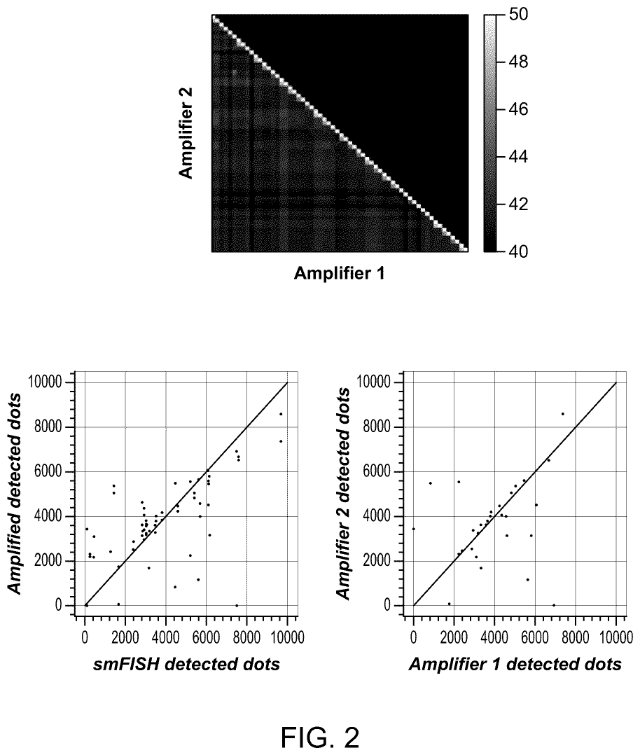

By preparing orthogonal sets of secondary, tertiary, and readout probe sequences, the signal amplification can be performed for many nucleic acid or protein species of interest both in cells, tissues, whole-mount samples, or extracted in vitro samples (FIG. 2). This amplification process for many targets can be achieved all together at once, because the sequences are orthogonal and do not cross-hybridize with each other (FIG. 2). The multiplexing amplification can be implemented with either nonbarcoded seqFISH, where the individual targets are imaged sequentially one by one, or barcoded seqFISH, where the colors or pseudocolors assigned to individual targets change in multiple rounds of barcoded hybridization. For example, over 20,000 genes can be detected by 4 barcoding rounds with 20 pseudocolors each (20⁴=160,000).

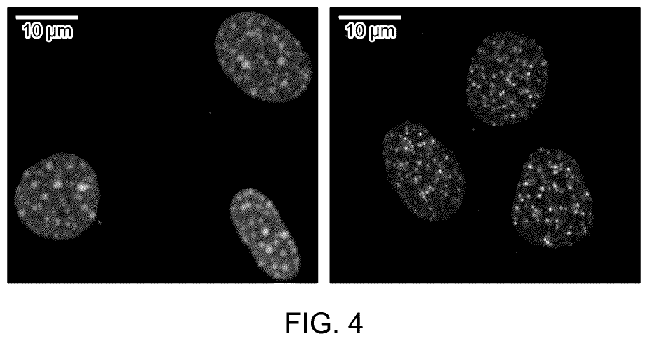

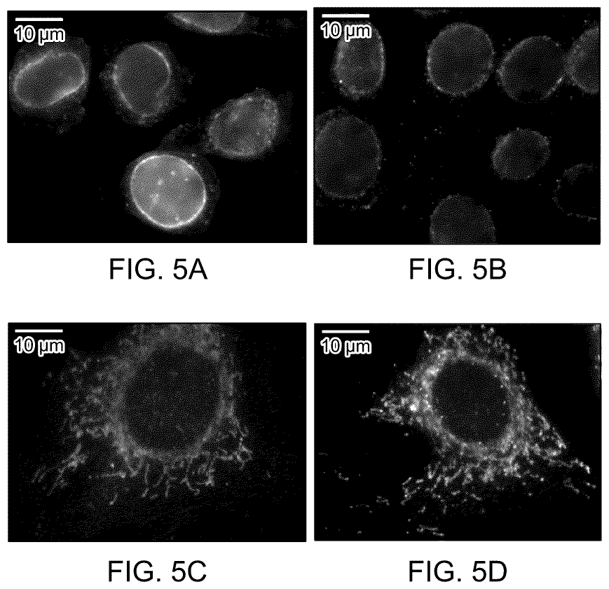

We have found good agreement between the dots detected by LANTERN with conventional smFISH, as well as between two different LANTERN amplifiers (FIG. 2, center and right and FIG. 3). In addition to RNA FISH, LANTERN can be used to amplify genomic DNA FISH signal (FIG. 4) and protein signal from DNA-conjugated antibodies (FIG. 5). Furthermore, we have applied LANTERN to amplify RNA FISH signal in multiple tissue and cell types such as mouse brain, human breast cancer biopsy, and whole-mount chicken embryo (FIG. 6). LANTERN is highly compatible with polyacrylamide hydrogel-embedding protocols and can be performed before or after embedding and clearing of a sample.

This method has the following advantages compared to existing amplification methods. LANTERN-amplified signals can be stably visualized across many rounds of readout probe hybridization and stripping because primary, secondary and tertiary probes are physically entangled (FIG. 1B). In contrast, signals from other amplification methods such as branched DNA amplification approaches could be reduced after several rounds of readout probe hybridization and stripping.

The level of amplification is determined by the number of rounds and the number of binding sites for successive rounds on the probes, unlike methods such as rolling circle amplification (RCA) and hybridization chain reaction (HCR), which are stochastic in nature.

Likely due to the stiff nature of dsDNA nanostructures, the amplified signal is highly stable over several rounds (FIG. 1). We found that after correcting for imperfect stage movement via image alignment, the center of highly amplified RNA FISH dots could be localized using Gaussian fitting to a root mean square precision of ˜3 nm in the X and Y directions across 12 rehybridizations, i.e. around 10 hours real time.

### REFERENCES

The following references are incorporated by their entirety.
Agouridas, V., El Mahdi, O., Diemer, V., Cargoët, M., Monbaliu, J. M., & Melnyk, O. (2019). Native Chemical Ligation and Extended Methods: Mechanisms, Catalysis, Scope, and Limitations. Chemical Reviews, 119(12). https://doi.org/10.1021/acs.chemrev.8b00712
Bauer, R. J., Zhelkovsky, A., Bilotti, K., Crowell, L. E., Evans, T. C., Jr, McReynolds, L. A., & Lohman, G. J. S. (2017). Comparative analysis of the end-joining activity of several DNA ligases. PloS One, 12(12), e0190062.
Bharti, A., Rashmi, T., Pankaj, A., & Rohit, B. (2017). Chemical Crosslinking: Role in Protein and Peptide Science. Current Protein & Peptide Science, 18(9), 946-955.
Bi, X., & Liang, A. (2016). Emerging Concepts in Analysis and Applications of Hydrogels. In Emerging Concepts in Analysis and Applications of Hydrogels. IntechOpen.
Braasch, D. A., & Corey, D. R. (2001). Locked nucleic acid (LNA): fine-tuning the recognition of DNA and RNA. Chemistry & Biology, 8(1). https://doi.org/10.1016/s1074-5521(00)00058-2
Chen, F., Tillberg, P. W., & Boyden, E. S. (2015). Expansion microscopy. Science, 347(6221), 543-548.
Chen, F., Wassie, A. T., Cote, A. J., Sinha, A., Alon, S., Asano, S., Daugharthy, E. R., Chang, J. B., Marblestone, A., Church, G. M., Raj, A., & Boyden, E. S. (2016). Nanoscale imaging of RNA with expansion microscopy. Nature Methods, 13(8).
doi.org/10.1038/nmeth.3899
Chen, H., Du, F., Chen, G., Streckenbach, F., Yasmeen, A., Zhao, Y., & Tang, Z. (2014). Template-directed Chemical Ligation to Obtain 3′-3′ and 5′-5′ Phosphodiester DNA Linkages. Scientific Reports, 4(1), 1-6. Cross-link. (n.d.). Retrieved May 4, 2021, from https://en.wikipedia.org/wiki/Cross-link
El-Sagheer, A. H., & Brown, T. (2017). Single tube gene synthesis by phosphoramidate chemical ligation. Chemical Communications, 53(77), 10700-10702.
Eng, C. H. L., Lawson, M., Zhu, Q., Dries, R., Koulena, N., Takei, Y., Yun, J., Cronin, C., Karp, C., Yuan, G. C., & Cai, L. (2019). Transcriptome-scale super-resolved imaging in tissues by RNA seqFISH. Nature, 568(7751), 235-239.
Eng, C. H. L., Shah, S., Thomassie, J., & Cai, L. (2017). Profiling the transcriptome with RNA SPOTs. Nature Methods, 14(12), 1153-1155.
Ghesquière, J., Gauthier, N., De Winter, J., Gerbaux, P., Moucheron, C., Defrancq, E., & Kirsch-De, M. A. (2012). Photocrosslinking between peptide-peptide or peptide-oligonucleotide by Ru(II)-TAP complexes. Chemistry, 18(1).
https://doi.org/10.1002/chem.201101458
Haque, M. M., & Peng, X. (2013). DNA-associated click chemistry. Science China. Chemistry, 57(2), 215-231.
Jin, J., Vaud, S., Zhelkovsky, A. M., Posfai, J., & McReynolds, L. A. (2016). Sensitive and specific miRNA detection method using SplintR Ligase. Nucleic Acids Research, 44(13). https://doi.org/10.1093/nar/gkw399
Lubeck, E., Coskun, A. F., Zhiyentayev, T., Ahmad, M., & Cai, L. (2014). Single-cell in situ RNA profiling by sequential hybridization. Nature Methods, 11(4), 360-361.
Nichols, N. M., Tabor, S., & McReynolds, L. A. (2008). RNA ligases. Current Protocols in Molecular Biology/Edited by Frederick M. Ausubel . . . [et Al.], Chapter 3. https://doi.org/10.1002/0471142727.mb0315s84
Nowak-Karnowska, J., Zielińska, K., Milecki, J., & Skalski, B. (2021). Thermally reversible and irreversible interstrand photocrosslinking of 5-chloro-2′-deoxy-4-thiouridine modified DNA oligonucleotides. Organic & Biomolecular Chemistry, 19(6), 1292-1295.
Pellestor, F., & Paulasova, P. (2004). The peptide nucleic acids (PNAs), powerful tools for molecular genetics and cytogenetics. European Journal of Human Genetics: EJHG, 12(9), 694-700.
Shah, S., Lubeck, E., Schwarzkopf, M., He, T. F., Greenbaum, A., Sohn, C. H., Lignell, A., Choi, H. M. T., Gradinaru, V., Pierce, N. A., & Cai, L. (2016). Single-molecule RNA detection at depth by hybridization chain reaction and tissue hydrogel embedding and clearing. Development, 143(15), 2862.
Shah, S., Lubeck, E., Zhou, W., and Cai, L. (2016). In Situ Transcription Profiling of Single Cells Reveals Spatial Organization of Cells in the Mouse Hippocampus. Neuron, 92(2), 342-357.
Shah, S., Takei, Y., Zhou, W., Lubeck, E., Yun, J., Eng, C. H. L., Koulena, N., Cronin, C., Karp, C., Liaw, E. J., Amin, M., & Cai, L. (2018). Dynamics and Spatial Genomics of the Nascent Transcriptome by Intron seqFISH. Cell, 174(2), 363-376.e16.
Shuman, S. (2009). DNA Ligases: Progress and Prospects. The Journal of Biological Chemistry, 284(26), 17365.
Tillberg, P. W., Chen, F., Piatkevich, K. D., Zhao, Y., Yu, C. C. (jay), English, B. P., Gao, L., Martorell, A., Suk, H. J., Yoshida, F., DeGennaro, E. M., Roossien, D. H., Gong, G., Seneviratne, U., Tannenbaum, S. R., Desimone, R., Cai, D., & Boyden, E. S. (2016). Protein-retention expansion microscopy of cells and tissues labeled using standard fluorescent proteins and antibodies. Nature Biotechnology, 34(9), 987-992.
Yang, B., Treweek. J. B., Kulkarni. R. P., Deverman. B. E., Chen, C. K., Lubeck, E., Shah, S., Cai. L., & Gradinaru, V. (2014). Single-Cell Phenotyping within Transparent Intact Tissue Through Whole-Body Clearing. Cell. 158(4). 945.

1. A composition for linked amplification tethered with exponential radiance, comprising a plurality of probes, wherein the composition comprises:
(i) one or more primary probes capable of binding one or more targets, wherein each primary probe comprises one or more secondary probe binding sites and optionally one or more readout probe binding sites;
(ii) one or more secondary probes, each capable of binding the primary probe, wherein each secondary probe comprises one or more tertiary probe binding sites or one or more readout probe binding sites;
(iii) optionally, one or more tertiary probes, each capable of binding to the secondary probe, wherein each tertiary probe comprises one or more quaternary probe binding sites or one or more readout probe binding sites;
(iv) optionally, one or more quaternary probes, each capable of binding to the tertiary probe, wherein each quaternary probe comprises one or more readout probe binding sites;
(v) one or more readout probes capable of binding to a readout probe binding site on the one or more primary, secondary, tertiary, or quaternary probes and capable of being detected; and
(vi) one or more molecules capable of stabilizing one or more primary, secondary, tertiary, or quaternary probes when or after the probe is hybridized or after the probe is hybridized.

2. (canceled)

3. A method for linked amplification tethered with exponential radiance, comprising steps of:
(i) contacting a sample with one or more primary probes that bind one or more targets, wherein each primary probe hybridizes to a target;
(ii) hybridizing one or more secondary probes to the primary probes; wherein each secondary probe comprises one or more tertiary probe binding sites or one or more readout probe binding sites;
(iii) optionally, hybridizing one or more tertiary probes to at least one secondary probe, wherein each tertiary probe comprises one or more quaternary probe binding sites or one or more readout probe binding sites;
(iv) optionally, hybridizing one or more quaternary probes to at least one tertiary probe, wherein each quaternary probe comprises one or more readout probe binding sites; and
(v) stabilizing one or more primary, secondary, tertiary, or quaternary probes during or after steps (i)-(iv);
(vi) hybridizing readout probes capable of detection to the one or more readout probe binding sites;
(vii) imaging the cell after step (vi) so that the interaction of the primary probe to the nucleic acids is detected; and
(viii) optionally repeating the contacting and imaging steps, each time with a new plurality of detectably labeled readout probes, wherein at least one readout probe for one target differs from at least one other readout probes for the same target in their detectable moieties, so that a target in the sample is described by a barcode, and can be differentiated from another target in the sample by a difference in their barcodes.

4. The method of claim 3, wherein any of the steps (i)-(vii) are repeated either individually or in any combination thereof.

5. The method of claim 3, comprising stabilizing the primary probe, secondary probe, tertiary probe, or quaternary probe.

6-9. (canceled)

10. The method of claim 3, wherein the secondary, secondary and tertiary, secondary and tertiary and quaternary, secondary and quaternary, tertiary, tertiary and quaternary, or quaternary probes comprise at least two amplifier fragments.

11. The method of claim 10, wherein the secondary probe amplifier fragments comprise at least:
(a) a first amplifier fragment, wherein the first amplifier fragment comprises a region of complementarity to the primary probe, and wherein the region of complementarity hybridizes to the primary probe; and
(b) a second amplifier fragment, wherein the second amplifier fragment comprises a region of complementarity to the primary probe, and wherein the region of complementarity hybridizes to the primary probe.

12. The method of claim 10 wherein the tertiary probe amplifier fragments comprise at least:
(a) a first amplifier fragment, wherein the first amplifier fragment comprises a region of complementarity to a secondary probe or to the first or second amplifier fragment of the secondary probe, and wherein the region of complementarity hybridizes to the secondary probe or to the first or second amplifier fragment of secondary probe; and
(b) a second amplifier fragment, wherein the second amplifier fragment comprises a region of complementarity to the secondary probe or to the first or second amplifier fragment of secondary probe, and wherein the region of complementarity hybridizes to the secondary probe or to the first or second amplifier fragment of secondary probe.

13. The method of claim 10 wherein the quaternary probe amplifier fragments comprise at least:
(a) a first amplifier fragment, wherein the first amplifier fragment comprises a region of complementarity to a tertiary probe or to the first or second amplifier fragment of the tertiary probe, and wherein the region of complementarity hybridizes to the tertiary probe or to the first or second fragment of tertiary probe; and
(b) a second fragment, wherein the second fragment comprises a region of complementarity to the tertiary probe or to the first or second amplifier fragment of the tertiary probe, and wherein the region of complementarity hybridizes to the tertiary probe or to the first or second fragment of tertiary probe.

14. The method of claim 3 wherein a ligase ligates the first or second fragments of any secondary, tertiary, or quaternary amplifier fragments.

15. The method of claim 3 wherein a splint sequence hybridizes to the first amplifier fragment, the second amplifier fragment, or both amplifier fragments of the secondary, tertiary, or quaternary amplifier fragments, and wherein the splint sequence comprises at least two splint sequence fragments.

16. The method of claim 3 wherein the splint sequence fragments are ligated.

17. The method of claim 3 wherein a readout probe hybridizes to either the first or second amplifier fragment of the secondary, tertiary, or quaternary probes.

18. The method of claim 3 wherein a readout probe hybridizes to a splint sequence fragment.

19. The method of claim 3, wherein the stabilizing is selected from the group consisting of enzyme ligation, chemical ligation, UV crosslinking with or without oligo splint probes, hybridization of splint probes, crosslinking through a matrix, and chemical crosslinking, and any combination thereof.

20. The method of claim 3 wherein the targets are selected from transcripts, RNA, DNA loci, chromosomes, DNA, proteins, lipids, glycans, cellular targets, organelles, and any combinations thereof.

21. The targets of claim 20, wherein the targets are conjugated to one or more oligonucleotide sequences.

22. The method of claim 3 wherein the primary, secondary, tertiary, or quaternary probes stabilized by cis-ligation.

23-24. (canceled)

25. The method of claim 3 claims, wherein the primary, secondary, tertiary, or quaternary probes stabilized by cis-ligated during the contacting or hybridizing of probes.

26. The method of claim 3 wherein each primary probe comprises a nucleic acid sequence complementary to a target nucleic acid sequence.

27-28. (canceled)

29. The method of claim 3 wherein the primary, secondary, tertiary, or quaternary probes are cis-ligated by reactive groups on the probe wherein the reactive groups are a reactive pair selected from alkenes, alkynes, azides, amides, amines, nitrones, phosphates, tetrazines, and tetrazoles.

30. The method of claim 3, wherein one or more of the readout probes comprise an oligonucleotide or antibody with a detectable moiety.

31. The method of claim 3, wherein the readout probes comprise oligonucleotides with the same sequence.

32. The method of claim 3, wherein the readout probes comprise oligonucleotides with different sequences.

33-44. (canceled)

45. The method of claim 3, further comprising repeating the contacting and imaging steps, each time with a new plurality of detectably labeled readout probes, wherein in each new plurality at least one readout probe for one target differs from at least one readout probe for the same target in a previous plurality, wherein they differ at least in their detectable moieties.

46. (canceled)

47. The method of claim 3, wherein the primary, secondary, or tertiary probes are ligated or cross-linked, either cis or trans.

48-51. (canceled)
